(19) United States
(12) Patent Application Publication
(10) Pub. No.: US 2009/0143723 A1
(43) Pub. Date: Jun. 4, 2009

(54) FLOW CONTROL DEVICE FOR PERITONEAL DIALYSIS

(75) Inventors: Edward Szpara, Saint Charles, IL (US); Steven C. Jepson, Palatine, IL (US); Michael A. Gloss, Minneapolis, MN (US); Paul D. Rahn, Crystal Lake, IL (US); William R. Griswold, Bristol, WI (US); Ying-Cheng Lo, Green Oaks, IL (US)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Zurich (CH)

(21) Appl. No.: 11/947,261

(22) Filed: Nov. 29, 2007

(51) Int. Cl. A61M 1/28

(52) U.S. Cl. 604/29

(57) ABSTRACT

A peritoneal dialysis flow control device in one embodiment includes: (i) a first cap including a first medical fluid line connection and a second medical fluid line connection; (ii) a gasket mated with the first cap where the gasket defines a first aperture in fluid communication with a first port and a second aperture in fluid communication with a second port; and (iii) a second cap including a third medical fluid line connection where the second cap is sealed rotatably to the gasket.

### BACKGROUND

Two general types of dialysis therapy are now in wide spread use. One type, hemodialysis, provides for removing waste products by passing the blood of a patient through an appropriately constructed dialyzer unit. A second type of dialysis therapy, peritoneal dialysis, utilizes the membrane in a patient's peritoneal cavity for the purpose of separating waste products from the patient's fluid systems.

In one form of peritoneal dialysis, referred to as continuous ambulatory peritoneal dialysis (“CAPD”), dialysis fluid is introduced into the patient's peritoneal cavity by means of an in-dwelling peritoneal catheter. The dialysis solution is permitted to remain in the peritoneal cavity of the patient for about four to six hours. At the end of this time, spent fluid is drained from the patient's cavity, under the influence of gravity, and fresh dialysis fluid is infused into the cavity to continue the process.

The patient carries out the drain and fill cycle noted above by executing a predetermined sequence of steps to first drain spent fluid and then to refill the peritoneal cavity with fresh fluid. Carrying out the predetermined sequence of steps requires opening and closing, in a predetermined sequence, a plurality of flexible tubing members in a fluid flow transfer set connected between the external end of the patient's catheter and solution containers of peritoneal dialysis fluid.

Known CAPD systems require many clamping and unclamping steps for a single exchange. For example, one system requires the following manual steps:

1. patient connects to transfer set;
2. patient opens a twist clamp to drain the patient;
3. patient closes twist clamp after the drain cycle;
4. patient places and closes a clamp on the drain line;
5. patient breaks a frangible seal to the solution line;
6. patient opens the clamp on the drain line to start a flush cycle;
7. patient closes the clamp on the drain line to end the flush cycle;
8. patient opens a twist clamp on the patient line to start a fill cycle;
9. patient places and closes a clamp on the solution line to end the fill cycle; and
10. patient disconnects transfer set.

A need exists to simplify the above steps in a safe and reliable manner.

### SUMMARY

Various embodiments of an improved continuous ambulatory peritoneal dialysis (“CAPD”) device are provided. The devices control the flow of dialysate to and from the patient, e.g., from the solution bags and to one or more drain bags. The devices can stop the flow of dialysate at various steps in the therapy, e.g., to enable the user to safely disconnect from the device in the middle of therapy if needed. In an embodiment, the devices also prevent leakage after therapy, prior to the devices being discarded. The devices also seal the CAPD system and maintain sealing integrity. Certain features of the devices can also prevent the patient from going backwards during therapy, e.g., from a filling to a flushing step. To that end, the devices also sequence the patient through the steps of therapy and provide an indication of when the patient has advanced to the next step.

In a first primary embodiment, a flow control device is actuated via a lever. The flow control device includes two main components, namely, a lever and a base. The lever is removably and rotatably connected to the base. The lever includes an arm, which in an initial position is located at a clockwise distance away from a solution line. The solution line is connection in a Y-fashion to patient and drain lines. The solution line is fitted initially with a breakable or frangible seal. In the drain position, fluid is able to flow from the patient through the patient line, the Y-connection and the drain line to drain. When the drain cycle is complete, the patient rotates the lever arm to a flush position. In this step, the lever contacts the solution line and bends the solution line enough such that the frangible seal precluding flow of fresh fluid through the solution line is broken, thus allowing fresh fluid to flow through the solution line. The lever arm locks into place at the flush position. Here, a cam extending from a hub of the lever arm precludes the patient line. Therefore, the fresh solution is allowed to flow through the Y-connection and drain line to flush residual spent fluid from the tubing set. It is conceivable that the flush step can be eliminated if a sterile connection is made between the patient's transfer set and the patient line connector.

When flushing is complete, the patient rotates the lever further in the same direction to a fill position. Here, the lever is locked in the fill position, the cam that occludes the patient line is rotated off of the patient line and a second cam is rotated to occlude the drain line. The remainder of fresh solution from a solution bag is allowed to flow through the solution line, through the patient line to the patient.

When filling is complete, the patient moves the lever to a closed position, in which the lever is locked again in this closed position. Here, the cam that occludes the drain line is moved off of the drain line and a third cam is moved again to close the patient line. This protects the patient from contamination, while the fresh solution just delivered is allowed to dwell within the patient's peritoneum.

While the lever is illustrated herein as being operated manually, it is also contemplated to connect the lever to a motor and motor controller. For example, the motor can rotate the lever automatically (e.g., while the patient sleeps) according to a timer operating with the controller or via a manual pushbutton input, which signals the motor to rotate the lever to the next position.

In one implementation of this first primary embodiment, a peritoneal dialysis flow control device includes: a base configured to hold medical tubing; and a lever connected pivotally to the base along an axis of rotation, the lever including cams extending radially away from the axis, the cams spaced circumferentially away from each other such that the medical tubing is opened and occluded according to steps of a medical treatment as the lever is pivoted with respect to the base. The lever can include a handle for manual operation of the lever. A motor can be coupled to the lever so as to be able to move the lever relative to the base. The cams can be rounded to reduce a required tubing clamping force.

The base includes a bottom, the bottom having tubing holders, and sidewalls extending from the bottom, the sidewalls including a lever holder positioned along the axis of rotation. The base can also be configured to hold first, second and third lines of the tubing and to enable the first, second and third lines to be placed in fluid communication with each other. The base can further define a plurality of longitudinal lumens for accepting the tubing. When treatment is peritoneal dialysis, the base is configured such that the first, second and third lines can be solution, patient and drain lines, respectively, the steps of the medical treatment including drain, flush and fill steps of the peritoneal dialysis treatment.

The lever extends underneath a line of the tubing such that the line is moved when the lever is pivoted, the movement of the line causing a frangible seal within the line to break so that fluid can flow through the line. The lever can also define a plurality of ratchets spaced apart circumferentially about the axis of rotation at angles so that the cams of the lever occlude different areas of the tubing sequentially. The lever can further include a hub, the cams extending from the hub, each cam having a corresponding ratchet extending from the hub. One of the base and the lever includes at least one ratchet and the other of the base and lever includes at least one lock, the at least one ratchet and at least one lock configured to hold the lever in different positions in which a different one of the cams occludes a desired area of the tubing.

In another implementation of this first primary embodiment, a peritoneal dialysis flow control device includes: a base configured to hold a solution line, a patient and a drain line; a lever connected moveably to the base, the base and lever configured to: (i) allow spent fluid to flow from the patient, through the patient and drain lines, to a drain when the lever is in a first position relative to the base, (ii) allow fresh fluid to flow from a supply, through the solution and drain lines, to the drain when the lever is in a second position relative to the base, and (iii) allow fresh fluid to flow from the supply, through the solution and patient lines, to the patient when the lever is in a third position relative to the base.

The lever can be connected pivotally to the base, the lever set at different angles relative to the base to achieve the first, second and third positions. The first position of the lever is at least substantially parallel to the base. At least one of: (a) neither the patient line nor the drain line is occluded when the lever is in the first position, (b) the patient line is occluded when the lever is in the second position, and (c) the drain line is occluded when the lever is in the third position. The device can include a fourth position, the lever moved to the fourth position relative to the base after the fresh fluid has been delivered to the patient. The device can further be configured to rupture a frangible seal within the solution line when the lever is moved from the first to the second position. The base and lever can include interacting apparatuses to maintain the lever in at least one of the first, second and third positions.

In a further implementation of this first primary embodiment, a flow control device for peritoneal dialysis conducted using an aseptic connection between a patient and a patient includes: a base configured to hold the patient line, a solution line, and a drain line; a lever connected moveably to the base, the base and lever configured to: (i) allow spent fluid to flow from the patient, through the patient and drain lines, to a drain when the lever is in a first position relative to the base, and (ii) allow fresh fluid to flow from a supply, through the solution and patient lines, to the patient when the lever is in a second position relative to the base.

At least one of: (a) neither the patient line nor the drain line is occluded when the lever is in the first position; (b) the first position is a home position that does not require movement of the lever with respect to the base; (c) an intermediate flush position exists between the first and second positions, wherein the patient line is occluded, and which allows fresh fluid to flow from the supply, through the solution and drain lines; and (d) the drain line is occluded and the patient line is open when the lever is in the second position.

The device in this third implementation of the first primary embodiment can include a third position, the lever moved to the third position relative to the base after the fresh fluid has been delivered to the patient. The patient line is occluded and the drain line is open when the lever is in the third position. The flow control device can be configured to rupture a frangible seal within the solution line when the lever is moved from the first position to the second position. The base and lever can also include interacting apparatuses to maintain the lever in at least one of the first and second positions.

In a second primary embodiment, a flow control device is actuated via a base and accompanying dial that rotates with respect to the base. The dial in an embodiment is snap-fitted in rotational engagement with the base. The base includes openings to accept tubing of a tubing set. The tubing set is modified to include an additional piece of tubing that connects to a Y-connector outside of the flow control device to enable the drain line and solution line to tie together into the patient line. In an embodiment, the patient line outlet of the Y-connector is connected directly to a connector that connects to the patient's transfer set. The dial includes indicia and the base includes an indicator, so that the patient knows where to turn the dial in relation to the base for performing a particular cycle of the treatment. In this manner, all of the solution from the solution bag can fill the patient's peritoneum. After the patient fill, the fresh solution is allowed to dwell within the patient, after which the above cycles are repeated.

In one implementation of this second primary embodiment, a peritoneal dialysis flow control device includes: a base configured to hold medical tubing, the base including at least one pressure plate configured to be positioned adjacent to at least one of first and second lines of the tubing when the tubing is loaded into the base; and a dial connected to the base rotatably about an axis, the dial including at least one cam operable with the at least one pressure plate, the at least one cam spaced radially away from the axis, such that at least one of the first and second lines is occluded or opened according to steps of a medical treatment as the dial is rotated with respect to the base.

The flow control of this second primary embodiment can include a plurality of the cams provided on a bottom side of the dial, a top side of the dial including a raised portion for grasping and turning the dial. The dial can be snap-fitted rotatably to the base. The medical tubing in one embodiment is for peritoneal dialysis, the first line being a drain line, the second line being a solution line. The base can define openings through which the first and second lines can pass.

The flow control device can be configured such that the dial is turned in a first direction for a first step of the medical treatment and turned in a second direction for a second step of the medical treatment. The first step can be a drain step and the second step is a fill step, and which includes a flush step performed between the drain step and the fill step, the base and dial providing support against which to break a frangible seal in the medical tubing, so that fresh solution flows to perform the flush step while the dial is turned in the first direction.

The dial can display indicia for breaking the frangible seal adjacent the frangible seal while the dial is turned in the first direction. Here, the first step can be a drain step and the second step is a fill step, wherein neither the first line nor the second line is occluded by the dial during the drain step and a line leading to drain is occluded by the dial during the fill step. A flush step can be performed between the drain step and the fill step, wherein neither of the first and second lines is occluded by the dial during the flush step.

The flow control device can include at least one of: (a) a tactile feedback producing apparatus to signal when one of the first and second lines is opened or occluded after the dial has been rotated with respect to the base, and (b) an indicating apparatus provided on the base and the dial to indicate where to rotate the dial with respect to the base. At least one of: (i) the indicating apparatus on the base includes directional indication and (ii) the indicating apparatus on the dial includes printed indicia.

In another implementation of this second primary embodiment a peritoneal dialysis flow control device for peritoneal dialysis includes: a base configured to hold a drain line and a solution line of a peritoneal dialysis tubing set, the base including a pressure plate configured to be positioned adjacent to the drain line when the tubing set is loaded into the base; and a dial connected to the base rotatably about an axis, the dial including at least one cam operable with the pressure plate, such that the drain line is opened or occluded according to peritoneal dialysis steps by rotating the dial with respect to the base. At least one cam occludes the drain line when a fill cycle is indicated by the dial.

In a further implementation of this second primary embodiment a peritoneal dialysis flow control device for peritoneal dialysis includes: a tubing set having a drain line, a solution line and a patient line; a base configured to hold the drain line and solution line of the tubing set, the base including a pressure plate configured to be positioned adjacent to the drain line when the tubing set is loaded into the base; and a dial connected to the base rotatably about an axis, the dial including at least one cam operable with the pressure plate, such that the drain line is opened or occluded according to peritoneal dialysis steps by rotating the dial with respect to the base.

The solution line in this further implementation can include a frangible seal. The patient line can include a connector for connecting to a transfer set connected to a peritoneal dialysis patient. The device can include a Y-connection in which the drain line and the solution line communicate with the patient line. The at least one cam occludes the drain line when a fill cycle is indicated by the dial.

In a third primary embodiment, a twist clamp flow control device includes dual twisting occluders that bend or crimp one or more of the tubes during a particular cycle for treatment. Here, the flow control device includes a base to which the dual rotating clamping mechanism is connected. The base defines ribs that provide holes to accept the tubes of the tubing set. The ribs also define an opening that accepts a rod or support member of the dual occluding clamping mechanism. The clamping mechanism at each end has a rotatable clamp or tab that the patient can twist individually in one direction to occlude a particular tube while allowing flow through another tube. The occluded tube is occluded against an end or edge of the base. The clamping mechanism is twisted in a second direction to produce a different tubing state for a different cycle of therapy.

In one embodiment, the tubing set includes a Y-configuration such that a single patient line exits one end of the twist clamp flow control device while two tubes exit the other end of the flow control device. Here, the patient can turn the clamp associated with the single tube either way to occlude that tube. On the end having two extending tubes, the patient turns the associated twist clamp in one direction to occlude one of the tubes (and allow the second tube to be opened) and in the other direction to alternatively occlude the other of the tubes (and allow the first tube to be opened). Suitable indicia is provided on the twist clamps to direct the patient which direction to turn which clamp for each cycle.

In one implementation of this third primary embodiment a peritoneal dialysis flow control device includes: a body configured to accept a tubing set, the body including a first portion against which a first tube of the tubing set rests and a second portion against which a second tube of the tubing set rests; a first occluding member connected rotatably to the body, the first occluding member rotatable towards the first portion of the body to occlude the first tube and away from the first portion to open the first tube; and a second occluding member connected rotatably to the body, the second occluding member rotatable towards the second portion of the body to occlude the second tube and away from the second portion to open the second tube. The first and second portions can be first and second ends of the body, the first and second occluding members positioned to kink the first and second tubes against the first and second ends. One of the first and second tubes is a solution tube, and wherein the body is shaped to angle the solution tube away from the other of the first and second tubes to allow for proper flow of fluid from the solution tube to the other of the first and second tubes.

The device in the first implementation of the third embodiment can include a third portion against which a third tube of the tubing set rests, and wherein one of the first and second occluding members is further rotatable towards the third portion of the body to occlude the third tube and away from the third portion to open the third tube. The first and second occluding members can be connected rotatably to a rod, the rod connected to the body. The dialysis flow control device can include at least one of: (i) a tactile feedback producing apparatus configured to signal when one of the first and second tubes is occluded after the first or second occluding member has been rotated with respect to the first or second portion, respectively, and (ii) a locking apparatus configured to lock the first or second occluding member after it has been rotated to occlude the first or second tube.

In another implementation of this third primary embodiment a peritoneal dialysis flow control device includes: a body configured to accept a tubing set, the body including a first end and a second end; a first occluding member connected moveably to the body, the first occluding member moveable to kink and unkink a first tube of the tubing set against the first end; and a second occluding member connected moveably to the body, the second occluding member moveable to kink and unkink a second tube of the tubing set against the second end. The first and second occluding members can be connected rotatably to the body. The tubing set can include a third tube, and wherein one of the first and second occluding members is further moveable to kink and unkink the third tube. The first and second occluding members can also be connected moveably to a rod, the rod connected to the body.

The first and second occluding members can each include at least one of: (i) a tapered edge configured to provide an increasing kinking force to the first or second tube against the first or second edge, respectively, as the respective member is moved; (ii) a face that remains in kinking contact with first or second tube, respectively, when the member is moved to a certain point relative to the base; and (iii) a tactile feedback producing apparatus configured to signal when one of the first and second tubes is occluded after the first or second occluding member has been moved into kinking contact with the first or second tube, respectively.

In a further implementation of this third primary embodiment a peritoneal dialysis flow control device includes: a body configured to accept a solution tube, patient tube and drain tube used for peritoneal dialysis; a first occluding member connected moveably to the body, the first occluding member moveable to kink and unkink the patient tube; and a second occluding member connected moveably to the body, the second occluding member moveable to kink and unkink the solution and drain lines.

In the flow control device of this implementation of the primary third embodiment, the second occluding member can be moveable in a first direction to kink the solution line and in a second direction to kink the drain line. The second occluding member can be moveable in the first direction to unkink the drain line and in the second direction to unkink the solution line. The body can be shaped to angle the solution tube away from the patient and drain tubes to allow for proper flow of fluid from the solution tube to the patient and drain tubes. The solution tube can include a frangible seal and the second occluding member is positioned to break the frangible seal when moved.

In a fourth primary embodiment, a flow control device is actuated via a three-piece unit including a two-port cap, a one-port cap and a gasket. The two-port cap is rotatable with respect to the one-port cap and vice-versa and, in one embodiment, the two-port cap is snap-fitted in rotational engagement with the one-port cap. The gasket is fitted between the two-port cap and the one-port cap, e.g., onto one of the caps and engaging the other cap.

The top of the two-port cap and the top of the one-port cap include or define ports, e.g., luer or tube fittings, that accept the various CAPD tubes. In one embodiment, the one-port cap includes or defines a port that sealingly accepts the patient line, while the two-port cap includes or defines ports that sealingly accept the drain and solution lines, respectively. This device eliminates the Y-tubing connection between the three lines.

The gasket in one embodiment fits sealingly onto and moves with the two-port cap. The gasket defines a pair of outwardly extending annular ribs that snap-fit in a rotatably sealed manner over an inwardly extending annular rib of the one-port cap. This seals the one-port cap to the two-port cap but allows both caps to rotate with respect to each other. The two-port cap also defines an inwardly extending annular groove that rotatably accepts a second inwardly extending annular rib of the one-port cap. This engagement also allows the two-port cap to rotate in a sealed manner with respect to the one-port cap.

The inside of the top of the one-port cap defines an elongated fluid path groove or slot. A patient line lumen (defined by the patient line port) extends through the bottom of the one-port cap. Drain and solution line lumens (defined by the drain and solution line ports) extend through the top of the two-port cap. The gasket also defines a fluid path slot and a pair of circular fluid openings, one for the solution line and one for the drain line, which are connected to the two-piece cap. The openings in an embodiment are circumscribed by a circular grommet or raised seal that seals to the inside of the top of the one-port cap around the patient line opening as the various gasket holes (and in certain cases corresponding solution and drain openings of the two-piece cap) are rotated into fluid communication with the patient opening of the one-piece cap. The slot is provided for an all-lines-open state, which allows fluid to flow through each of the lines, e.g., to provide a final drain of all of the bags.

The gasket also provides areas having raised circular sealing rings. The sealing areas come into communication with the inside surface of the top of the one-port cap, around the patient line opening, at various times to help seal the patient line in a closed position. The sealing areas also allow the flow control device to have an all-lines-closed state, e.g., during patient dwell.

Once the user connects the lines to the flow control device and the patient line to the patient's transfer set, therapy can begin. In the United States (“U.S.”), therapy begins with a vented position in which the solution line is allowed to communicate with the drain line and drain bag. This is required for steam sterilization. Otherwise, e.g., if the solution line is closed at the solution bag by a frangible closure and closed at the flow control device by its internal seals, the tubing would collapse during steam sterilization. Collapsed tubing impacts the flow performance of the system. The U.S. version of the three-piece dial device accordingly includes an extra (sixth) step, the first being the venting step in which the patient does not rotate either cap with respect to the other. When the patient opens a new disposable package, the first step in the U.S. therapy is for the patient to hold the two-port cap (and connected gasket) stationary, for example, and rotate the one-port or patient port cap in a direction, e.g., counterclockwise to a second or drain position, which allows the previous fill to drain via gravity from the patient to the drain bag (while the solution line is closed) to purge air from the system. Then, the patient rotates the patient-port cap in the same direction to a third, flush position and breaks a frangible seal in the solution line to allow fresh solution to flush the drain line (while the patient line is closed). Then, the patient rotates the patient-port cap in the same direction to a fourth, fill position, which allows fresh solution to gravity fill the patient's peritoneum (while the drain line is closed). After the fill is complete, the patient rotates the one-piece cap in the same direction to a fifth, all-lines-closed state, which isolates each of the lines until the patient disconnects from the control device (e.g., during a dwell phase in which the new dialysate dwells within the patient's peritoneum to remove waste and ultrafiltrate). The patient then disconnects the patient line from the transfer set and turns the patient-port cap in the same direction to a sixth, all-lines-open state, in which all three lines are opened to allow any remaining solution to run from the drain bag and the solution bag through the patient line to a house drain (e.g., toilet).

The European version is virtually the same as the U.S. version except that the first venting step is not performed. Also disclosed herein is a flow control device that operates with a CAPD system that does not need a drain bag.

In implementation of this fourth primary embodiment, a peritoneal dialysis flow control device includes: a first cap including a first medical fluid line connection and a second medical fluid line connection; a gasket mated with the first cap, the gasket defining a first aperture in fluid communication with the first medical fluid line connection and a second aperture in fluid communication with the second medical fluid line connection; and a second cap including a third medical fluid line connection, the second cap sealed rotatably to the gasket mated to the first cap.

At least one of the first, second and third medical fluid line connections includes a port configured to receive a medical fluid line. One of the gasket and the second cap includes a double-ribbed projection and the other of the gasket and the second cap includes single-ribbed projection, the single-ribbed projection fitting sealingly and rotatably between ribs of the double-ribbed projection. One of the first cap and the second cap can include a locking device and the other of the first cap and the second cap can include at least one locking feature, the locking feature mating with the locking device to releasably secure the second cap to the first cap at a desired relative position. The mating of the locking feature with the locking device is configured to provide at least one of: (i) tactile feedback; (ii) audible feedback; (iii) overtravel protection; and (iv) anti-reverse protection.

The gasket can define at least one blind seal to seal the third medical fluid line connection when the third medical fluid line connection is rotated into alignment with the blind seal. Alternatively, the gasket defines at least one blind passageway, a portion of the passageway communicating with the third medical fluid line connection when the third medical fluid line connection is rotated into alignment with the blind passageway portion.

The second cap can define at least one blind passageway, the passageway communicating with at least one of the first and second medical fluid line connections when the at least one medical fluid line connection is rotated into alignment with the blind passageway.

The flow control device is characterized by at least one of: (i) the first medical fluid line connection being a solution port; (ii) the second medical fluid line connection being a drain port; (iii) the third medical fluid line connection being a patient port; and (iv) one of the first and second medical fluid line connections cooperating with an apparatus positioned to aid a user to break a frangible seal in a line connected to the first or second medical fluid line connection. The flow control device is further characterized by at least one of: the first and second caps including a grasping apparatus sized and shaped to enable a user to rotate one of the first and second caps relative to the other of the first and second caps.

In another implementation of this fourth primary embodiment, a peritoneal dialysis flow control device includes: a first cap including a solution line connection and a drain line connection; and a second cap including a patient line connection, the second cap sealed rotatably to the first cap so as to enable (i) a first relative position of the second cap to the first cap, in which the patient line connection is in fluid communication with the drain line connection, (ii) a second relative position of the second cap to the first cap, in which the solution line connection is in fluid communication with the drain line connection, and (iii) a third relative position of the second cap to the first cap, in which the solution line connection is in fluid communication with the patient line connection.

In this second implementation of the fourth embodiment, the flow control device can include an initial position in which gas can be vented from within the first and second caps when the first and second caps are assembled. The device can include an additional relative position, in which the none of the solution, drain and patient line connections is in fluid communication with each other. The device can further include an additional relative position, in which each of the solution, drain and patient line connections is in fluid communication with another of the solution, drain and patient line connections. The device can still further include a gasket, the gasket sealing the second cap rotatably to the first cap.

Further, at least one of: (i) the solution line connection is blocked in the first relative position of the second cap to the first cap; (ii) the patient line connection is blocked in the second relative position of the second cap to the first cap; and (iii) the drain line connection is blocked in the third relative position of the second cap to the first cap. The flow control device can be configured such that the second cap is rotated in a same direction between the first and second relative positions and second and third relative positions.

In a further implementation of this fourth primary embodiment, a peritoneal dialysis flow control device includes: a first cap; a second cap sealed rotatably to the first cap; a solution line connection, a drain line connection and a patient line connection provided with the first and second caps; a first relative position of the second cap to the first cap, in which the patient line connection is in fluid communication with the drain line connection; a second relative position of the second cap to the first cap, in which the solution line connection is in fluid communication with the drain line connection; and a third relative position of the second cap to the first cap, in which the solution line connection is in fluid communication with the patient line connection.

In one embodiment, the first cap includes the solution line connection and the drain line connection, and the second cap includes the patient line connection. The device can include a forth relative position, in which none of the solution, drain and patient line connections is in fluid communication with another of the solution, drain and patient line connections, and a fifth relative position, in which each of the solution, drain and patient line connections is in fluid communication with another of the solution, drain and patient line connections.

In a fifth primary embodiment, a stopcock arrangement is provided, which includes an inner cylindrical valve that rotates within an outer cylindrical housing. Patient, solution and drain line ports extend from the outer cylindrical housing and attach to patient, solution and drain tubes respectively. This device also eliminates the Y-tubing connection between the three lines. An outer jacket surrounds the solution line port and provides a rigid structure against which the patient can bend the solution tube to readily break the frangible seal. The ports define lumens or apertures that extend through the wall of the housing to the valve.

The inner cylindrical valve includes or defines a handle that resides outside the top of the outer cylindrical housing. The housing is sealed rotatably to the valve such that liquid does not leak between the housing and the valve and so that the valve can rotate within the housing. The patient twists the handle to turn the valve to a desired position with respect to the housing. In one embodiment, before doing so, the patient breaks a tab that initially locks the inner valve in a beginning position with respect to the outer housing.

The valve can be configured in a number of ways. In one way, the valve is a solid cylindrical piece in which different flow paths are bores made through the solid piece. Here, the valve defines or includes volcano or raised rib seals about the ends of the bores to seal to an inner surface of the housing.

In another embodiment, the valve defines or includes raised rib spiral pathways that circumvent (e.g., horizontally, vertically and/or diagonally) part or all of the outer cylindrical wall of the valve extending from a first desired position to a second desired position. One or more vertical pathways can also be used. The raised ribs forming the pathways seal to the inner surface of the outer cylindrical housing. The ends (or midsection) of the pathways come into fluid communication with the tubing ports as the valve is rotated to one of its operating positions. This can allow for an at least substantially hollow valve, saving material and cost.

Further alternatively, a combination of both the through-hole pathways and the raised-rib pathways can be used to limit the size of the stopcock flow control device.

As before, the initial position for the stopcock device can allow certain lines to vent, e.g., for use in the U.S. After connecting the patient line to the transfer set, the patient breaks the holding tab which allows the valve to be rotated within the housing. The patient rotates the valve to a second position to drain the patient (solution line closed), in a same direction to a third position to flush the drain line (patient line closed), in the same direction to a fourth position to fill the patient (drain line closed), to a fifth position to close all lines and to a sixth position to open all lines and allow the drain and supply bag to be drained through the patient line. Again, the European version of the stopcock flow control device does not require the initial venting step or configuration.

The stopcock flow control device in one embodiment includes: a housing having first, second and third medical fluid line connections; and a valve fitted rotatably inside the housing, the valve including a first flow path configured to communicate with the first and third line connections, a second flow path configured to communicate with the first and second line connections, and a third flow path configured to communicate with the second and third line connections. At least one of the first, second and third medical fluid line connections includes a port configured to receive a medical fluid line. The first medical fluid line connection can be a drain port. The second medical fluid line connection can be a solution port. The third medical fluid line connection can be a patient port. One of the first and second medical fluid line connections cooperates with an apparatus positioned to aid a user to break a frangible seal in a line connected to the drain or solution port.

The valve can include a body, wherein at least one of the first, second and third flow paths extending within the body. The body can be at least substantially solid, the at least one flow path bored through the body, or at least substantially hollow, the at least one flow path formed as a tube extending through the body.

The valve can include a body, at least one of the first, second and third flow paths extending along an external surface of the body. The at least one flow path can include a continuous raised ridge forming a seal with an inner wall of the housing and/or can extend diagonally or vertically along the external surface of the body. The valve can be configured such that the valve is rotated in a same direction (i) from a position in which the first flow path is in communication with the first and third line connections to a position in which the second flow path is in communication with the first and second line connections, and (ii) from the position in which the second flow path is in communication with the first and second line connections to a position in which the third flow path is in communication with the second and third line connections.

The valve can be configured such that it can be rotated in sequence (i) to a position in which the first flow path is in communication with the first and third line connections (ii) to a position in which the second flow path is in communication with the first and second line connections, and (iii) to a position in which the third flow path is in communication with the second and third line connections. The valve can include at least one of a handle and a grommet, the grommet forming the first, second and third flow paths.

The housing can include an inwardly projecting seal around a mouth of at least one of the first, second and third medical fluid line connections, the seal configured to seal to the body about a mouth of the at least one flow path.

The stopcock flow control device can be configured such that the housing can be rotated with respect to the valve so that none of the first, second and third medical fluid line connections can communicate fluidly with any of the first, second and third flow paths. The housing and valve in one embodiment include mating apparatuses that are configured to provide at least one of: (i) tactile feedback; (ii) audible feedback; (iii) overtravel protection; and (iv) anti-reverse protection.

The stopcock flow control device in another embodiment includes: a housing having a plurality of medical fluid line connections; and a valve fitted rotatably inside the housing, the valve including a body and a plurality of flow paths extending within the body, at least one of the flow paths bending 180 degrees to enable an inline pair of the medical fluid line connections existing on a same side of the housing to communicate fluidly. The body can be any one of: (i) at least substantially solid, the flow paths bored through the body; and (ii) at least substantially hollow, the flow paths formed as tubes extending through the body. The housing can include an inwardly projecting seal around a mouth of each of the medical fluid line connections, the seals configured to seal to the body about a mouth of at least one of the flow paths.

The stopcock flow control device in another embodiment includes: a housing having a plurality of medical fluid line connections; and a valve fitted rotatably inside the housing, the valve including a body and a plurality of flow paths along an external surface of the body. The flow paths each include a continuous raised ridge forming a seal with an inner wall of the housing. One of the flow paths can extend diagonally and another vertically along the external surface of the body. The valve can include a grommet, the grommet forming the external surface of the body.

Each of the primary embodiments discussed herein also includes one or more tactile feedback producing devices that provides audible and/or tactile feedback so that the patient can know when a particular state has been achieved. Each device also holds itself releasably in the different state positions in one embodiment. Each device can also include indicia or markings to inform the patient visually when a particular flow control state has been reached.

It is therefore an advantage of the present disclosure to reduce the amount of setup and treatment steps for peritoneal dialysis, such as continuous ambulatory peritoneal dialysis (“CAPD”).

It is another advantage of the present disclosure to reduce the amount of torque that the patient needs to apply to break a frangible seal to the fresh solution.

It is a further advantage of the present disclosure to allow the frangible seal to be broken without having to work the seal back and forth.

It is yet another advantage of the present disclosure to provide a nonreversible and relatively mistake free CAPD flow control device.

It is still a further advantage of the present disclosure to provide a flow control device with relatively little installation needed.

It is still another advantage of the present disclosure to provide flow control devices to reduce or eliminate external clamps.

Moreover, it is an advantage of the present disclosure to structure the flow control devices so as to reduce an amount of clamping force that a patient needs to apply to clamp a line.

Still further, an advantage of the present disclosure is to provide a relatively low cost flow control device.

Yet a further advantage of the present disclosure is to provide a flow control device that is compatible with different CAPD system requirements, e.g., for different countries.

Still other advantages include ergonomic and ready manipulation of the devices, minimum flow capacity, effective flushing, drainage of the bag at the end of therapy, minimization of pinholes, maintenance of frangible seals until breaking time, minimization of kinked tubing and of force needed to be applied to the transfer set, ability to be sterilized, and minimization of potential to overtravel.

Additional features and advantages are described herein, and will be apparent from, the following Detailed Description and the figures.

### BRIEF DESCRIPTION OF THE FIGURES

FIG. 1 is a perspective assembly view of one primary embodiment for a flow control device for peritoneal dialysis according to the present disclosure, which employs a base and lever rotatable with respect to the base.

FIG. 2 is a front elevation view of the base according to one embodiment of the present disclosure.

FIG. 3 is a top plan view of the base of FIG. 2.

FIG. 4 is a side elevation view of the base of FIG. 2.

FIG. 5 is a bottom plan sectioned view taken along line V-V of FIG. 2.

FIG. 6 is a cutaway perspective view of the base according to one embodiment of the present disclosure.

FIG. 7 is a perspective view of the lever according to one embodiment of the present disclosure.

FIG. 8 is a front elevation view of the lever of FIG. 7.

FIG. 9 is a top plan view of the lever of FIG. 7.

FIG. 10 is a side elevation view of the lever of FIG. 7.

FIG. 11 is a front elevation view of one embodiment of the lever actuated flow control device according to the present disclosure, which includes the lever in a first position relative to a base.

FIG. 12 is a front elevation view of one embodiment of the lever actuated flow control device according to the present disclosure, which includes the lever in a second position relative to the base.

FIG. 13 is a front elevation view of one embodiment of the lever actuated flow control device according to the present disclosure, which includes the lever in a third position relative to the base.

FIG. 14 is a front elevation view of one embodiment of the lever actuated flow control device according to the present disclosure, which includes the lever in a forth position relative to the base.

FIG. 15 is a perspective assembly view of a second primary embodiment for a flow control device for peritoneal dialysis according to the present disclosure, which employs a base and a dial rotatable with respect to the base.

FIGS. 16A and 16B are exploded perspective views of the dial actuated flow control device shown in FIG. 15.

FIG. 17 is a top plan view of one embodiment of the dial actuated flow control device according to the present disclosure, which shows a drain configuration for the device.

FIG. 18A is a top plan view of one embodiment of the dial actuated flow control device according to the present disclosure, which shows a break frangible configuration for the device.

FIG. 18B is a top plan view of one embodiment of the dial actuated flow control device according to the present disclosure, which shows a flush configuration for the device.

FIG. 19 is a top plan view of one embodiment of the dial actuated flow control device according to the present disclosure, which shows a fill configuration for the device.

FIG. 20 is a perspective assembly view of a third primary embodiment for a flow control device for peritoneal dialysis according to the present disclosure, which employs a base and dual tube occluders, which are twistable with respect to the base.

FIG. 21 is a side sectioned view of the twist actuated flow control device, which includes the twistable occluders in a drain position.

FIG. 22 is a side sectioned view of the twist actuated flow control device, which includes the twistable occluders in a flush position.

FIG. 23 is a side sectioned view of the twist actuated flow control device, which includes the twistable occluders in a fill position.

FIG. 24 is a side sectioned view of the twist actuated flow control device, which includes the twistable occluders in a dwell position.

FIG. 25 illustrates an alternative arrangement for the flow control device of FIGS. 20 to 24.

FIG. 26 is a perspective assembly view of a fourth primary embodiment for a flow control device for peritoneal dialysis according to the present disclosure, which employs two caps that rotate with respect to each other, wherein one of the caps houses a gasket.

FIGS. 27A to 27C are various views of one embodiment for a gasket of the flow control device of FIG. 26.

FIGS. 28A to 28C are perspective views of the caps of the flow control device of FIG. 26 just prior to being assembled together.

FIG. 29 is a perspective view of the caps of the flow control device of FIG. 26 as assembled, wherein one of the caps is rotatable with respect to the other.

FIG. 30 is a section view of the caps of the flow control device of FIG. 26 as assembled, wherein one of the caps is rotatable with respect to the other.

FIGS. 31A and 31B are perspective views of a patient line cap for European and U.S. therapies, respectively.

FIGS. 32A to 32E are top plan views of the flow control device of FIG. 26 during different steps of a European CAPD therapy.

FIGS. 33A to 33E are front sectioned elevation views of the flow control device of FIG. 26 during different steps of FIGS. 32A to 32E.

FIGS. 34A to 34F are top plan views of the flow control device of FIG. 26 during different steps of a U.S. CAPD therapy.

FIG. 35 is a perspective assembly view of one implementation of a fifth primary embodiment for a flow control device for peritoneal dialysis according to the present disclosure, which includes a cylindrical valve and a concentric housing around the valve, wherein the valve can be rotated inside the housing to maneuver the device into different steps of the peritoneal dialysis treatment.

FIG. 36 is a sectioned perspective view illustrating the components of an alternative flow control device, similar to that of FIG. 35.

FIG. 37 is a perspective view of a valve component of the flow control device of FIG. 36.

FIGS. 38A and 38B are perspective views of a grommet component of the flow control device of FIG. 36.

FIG. 39 is a perspective view of a housing component of the flow control device of FIG. 36.

FIGS. 40A to 40C are perspective views of a sequence of operation of the flow control device of FIG. 36 during CAPD therapy.

FIG. 41 is a perspective view of an alternative valve/grommet configuration.

FIG. 42 is a plan view of one embodiment of a line set for use with the flow control devices described herein.

FIG. 43 is a plan view of another embodiment of a line set for use with the flow control devices described herein.

FIG. 44 is a plan view of a further embodiment of a line set for use with the flow control devices described herein and in which a drain bag is eliminated.

FIG. 45 is an alternative gasket from the ones shown in FIGS. 27A to 27C, and which is used with a flow control device configured for the removed drain bag set of FIG. 44.

FIGS. 46A to 46E show one embodiment of a flow control device operating with a CAPD system that operates without a drain bag in various stages of operation.

### DETAILED DESCRIPTION

### Lever Actuated Device

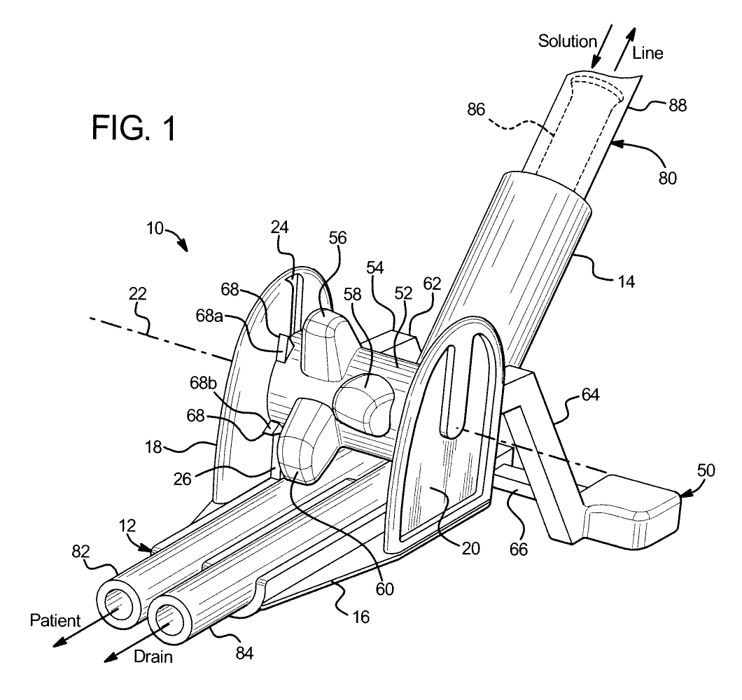

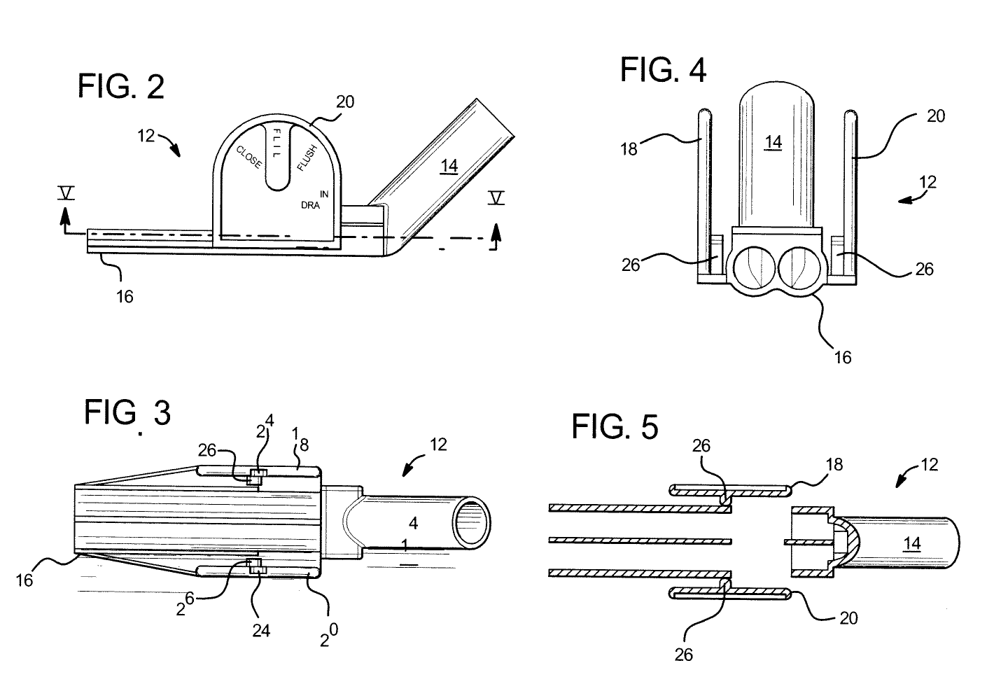

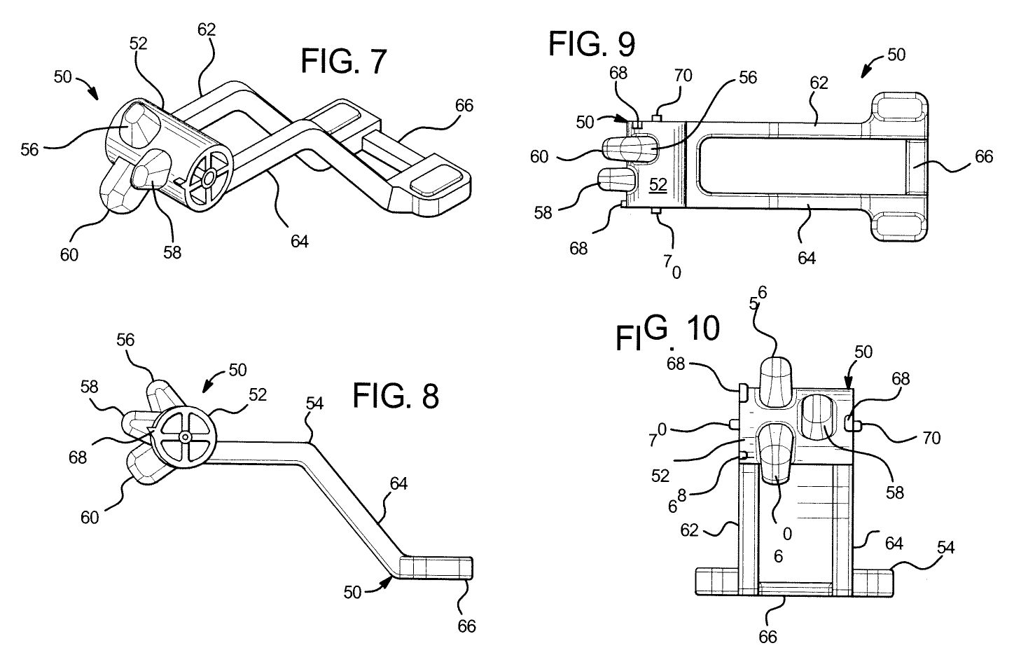

Referring now to the drawings and in particular to FIGS. 1 to 14, a first primary embodiment for a flow control device is a lever actuated device 10. Lever actuated device 10 includes three primary components, namely, a base 12, a lever 50 and peritoneal dialysis tubing set 80. Lever 50 rotates relative to base 12. Lever 50 includes a hub 52 and an arm 54 extending from hub 52. A number of cams, namely, cams 56, 58 and 60 extend from hub 52. Cams 56, 58 and 60 are spaced radially and axially along hub 52 so that different ones of cams 56, 58 and 60 contact and occlude either a patient line 82 or drain line 84 selectively and desirably as arm 54 of lever 50 is rotated manually with respect to base 12.

Cams 56, 58 and 60 in the illustrated embodiment include rounded contact surfaces. The rounded surfaces lessen an amount of clamping force needed to occlude patient line 82 or drain line 84 of tubing set 80.

Arm 54 includes a first extension 62 and a second extension 64, which straddle either side of solution line holder 14 of base 12. A rib 66 extends from the distal end of first extension 62 to the distal end of second extension 64. Rib 66 allows a patient or caregiver to grasp and move lever 50. Rib 66 also functions to break a frangible seal 86 located within solution line 88 of tubing set 80. FIGS. 42 to 45 illustrate that frangible seal 86 is provided at device 10 in one type of CAPD, e.g., European, setup. A North American setup places frangible seal 86 at the solution bag and not at device 10. For purposes of illustration, frangible seal 86 is shown.

Base 12 includes a bottom 16 from which solution line holder 14 extends. Sidewalls 18 and 20 also extend upwardly from bottom 16. Sidewalls 18 and 20 support pins 70 (not illustrated) extending from each side of hub 52, which define an axis of rotation 22 about which hub 52 and arm 54 of lever 50 rotate. Sidewalls 18 and 20 each define a groove 24 into which the pin is inserted and, for example, snapped fitted into a socket located at the intersection of axis of rotation 22 and the respective sidewall 18 or 20.

Base 12 and lever 50 are made of any suitable material, such as plastic, metal and combinations thereof. Suitable plastics include polypropylene, polycarbonate, polysulfone and polyethelene for example. Suitable metals include aluminum and stainless steel. In an embodiment, the material for hub 52 and that of pin 70 inserted into sidewalls 18 and 20 are selected such that friction between hub 52 and pin 70 is reduced. Suitable bearings and lubricants may also be used but are likely not necessary.

Hub 52 includes or defines angled ratchets 68. In the illustrated embodiment, a ratchet 68 is provided for each cam 56, 58 and 60. Ratchet 68 include a tapered face 68a and an at least substantially orthogonal face 68b. Tapered face 68a enables ratchet 68 to be turned in a counterclockwise manner with reference to the perspective view of FIG. 1 against a lock 26 extending upwardly and inwardly from each side 18 and 20 of base 12. Locks 26 are seen best in FIGS. 8, 9 and 10. Ratchets 68 lock hub 52 in multiple positions, each corresponding to a desired line occlusion state and according to a particular dialysis cycle, such as a drain, flush, fill and close cycle.

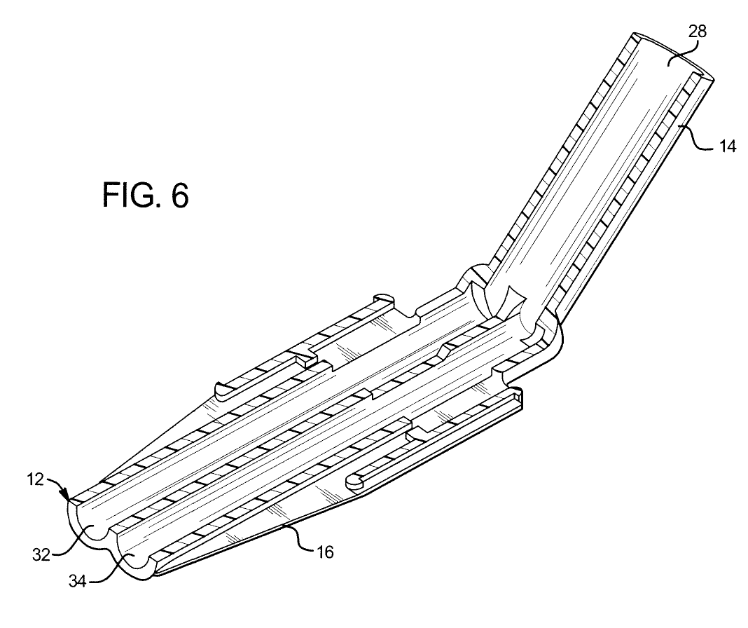

FIG. 6 is a sectioned view of base 12, showing sectioned bottom 16 and solution line holder 14. FIG. 6 illustrates that solution line holder 14 defines a tube lumen 28 that excepts and holds solution line 88 of tubing set 80. Bottom 16 in turn defines longitudinal lumens 32 and 34 that hold patient line 82 and drain line 84, which each tee into solution line 88. In the illustrated embodiment, lumen 28 is positioned at an angle relative to bottom 16 and lumens 32 and 34. The angle directs solution line 88 upward for example towards a solution bag that has a fixed elevation above fill control device 10. Patient line 82 connects to a patient's transfer set. Drain line 84 connects to a drain bag.

Although not shown, bottom 16 can include mounting holes that allow flow control device 10 to be attached fixedly to a tabletop or other fixture. Alternatively, bottom 16 can have an adhesive backing, which allows full control of device 10 to be adhered to a table or fixture. Further alternatively, a mounting bracket (not illustrated) separate from flow control device is mounted to a table or fixture, after which bottom 16 of flow control device 10 is slid into such bracket or otherwise removably connected to same.

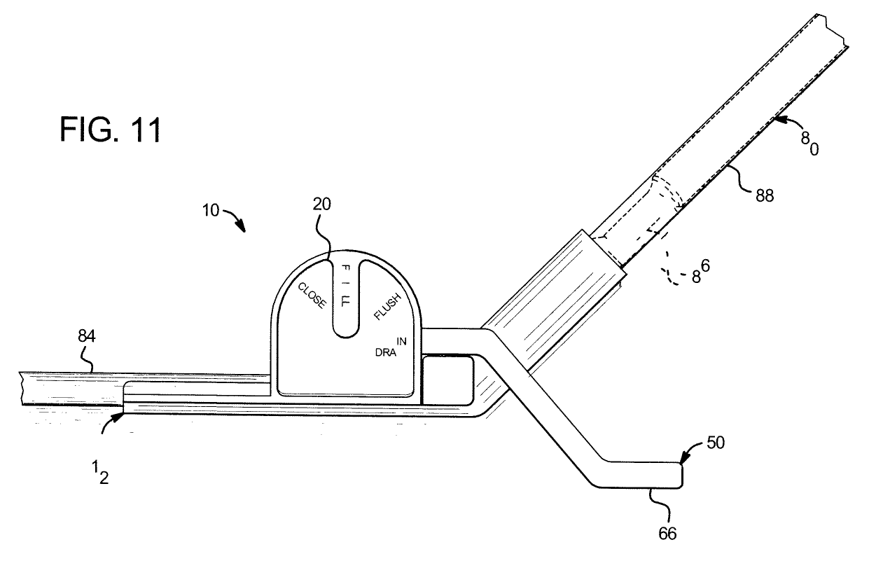

Referring now to FIGS. 1 and 11 through 14, the operation of flow control device 10 is illustrated. FIGS. 11 to 14 show that lever 50 can be placed in four positions relative to base 12, namely a drain position, a flush position, a fill position and a dwell position. These positions are marked for example on sidewall 20 and/or sidewall 18. As seen in FIGS. 1 and 11, in the drain position none of cams 56, 58 or 60 occludes either patient line 82 or drain line 84. Also, in a drain position lever 50 has not yet been pushed so that rib 66 has not yet moved past and broken frangible seal 86. Thus, fluid is allowed to flow from the patient, through the catheter implanted in the patient, to the transfer set connected to the patient, through patient line 82 of tubing set 80, around the Y-bend, which is currently sealed from solution line 88, out drain line 84 to a drain bag or house drain.

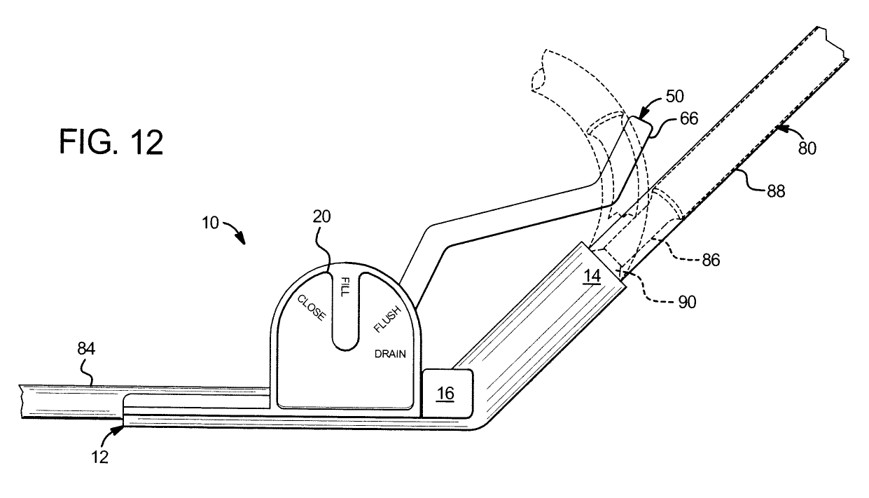

In FIG. 12, the patient moves lever 50 relative to base 12, such that rib 66 makes contact with solution line 88 of tubing set 80. As the patient pushes lever 50 further towards the flush position, frangible seal 86 is perforated or broken for example at pinch point 90. As lever 50 approaches the flush position, cam 60 occludes patient line 82, allowing fresh solution to flow from solution line 88, through drain line 84 and the Y-connection between lines 82, 84 and 88, which flushes or rinses tubing set 80 of residual spent dialysate from the drain cycle. When lever 50 reaches the flush position, ratchet 68 associated with cam 60 snaps past lock 26, so that if lever 50 is released by the patient, lever 50 remains held in the flush position. Here, tension applied by solution line 88, which is carried and moved along with rib 66 of lever 50 holds perpendicular face 68b of ratchet 68 firmly against lock 26.

If the connection between patient line 82 and the transfer set connected to the patient is an aseptic connection, the flush step may be eliminated. Here, lever 50 is moved directly from the drain position to the fill position. The procedure is reduced from a three-move procedure to a two-move procedure. In the two-move procedure, frangible seal 86 is broken when moving lever 50 from the drain position to the fill position. Removing the flush step simplifies device 10 by eliminating one of the cams and corresponding ratchets 68.

With the exception of the U.S. version, which requires the additional position of venting the solution line to the drain bag as discussed above, the devices herein typically start therapy with the in a drain position, which allows the patient to drain directly with no manipulation. The drain time is determined by the amount of fluid the patient may be holding. Once the patient identifies that draining is stopped, the patient performs a flush cycle. For flush, the patient can for example be required to leave to the device in the flush position for five seconds.

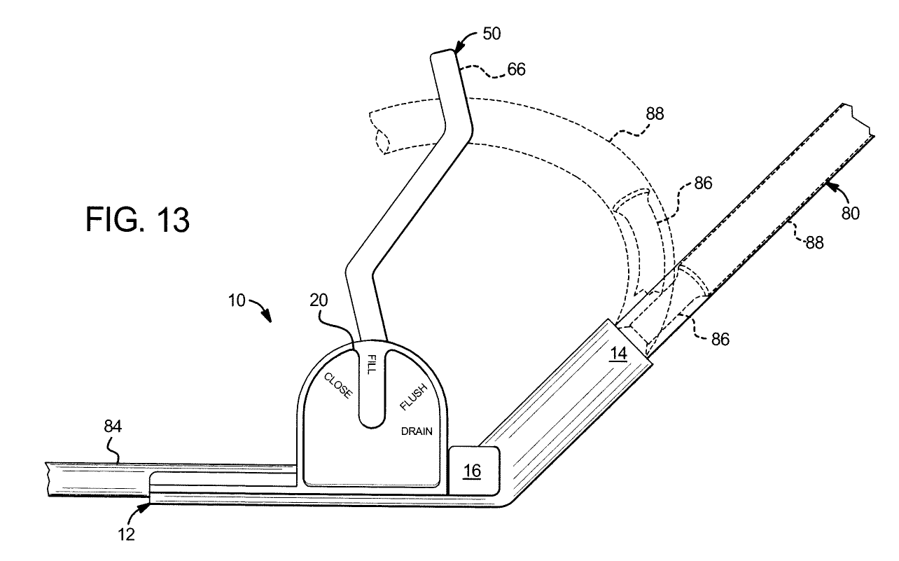

Referring now to FIG. 13, lever 50 is moved further counterclockwise to the fill position. Here, solution line 88 is carried even further by rib 66 of lever arm 54 upwardly and in the general counterclockwise direction of lever 50. At the fill position, ratchet 68 associated with cam 60 moves past and locks against lock 26. Cam 60, which occluded patient line 82 in the flush position, is rotated in a counterclockwise direction off of patient line 82 so that it now opens. Cam 58 in turn moves counterclockwise to occlude drain line 84. Now, fresh fluid flows through solution line 88 and patient line 82 to the patient, filling the patient's peritoneum with fresh dialysate. In an embodiment, lever 50 is left in the fill position until all or substantially all of the fresh dialysate is delivered to the patient.

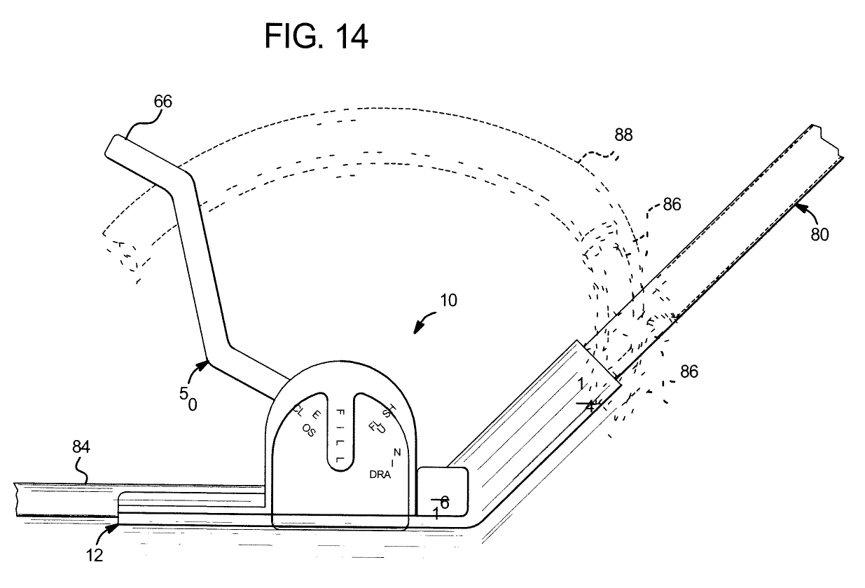

In FIG. 14, after the fresh solution is delivered to the patient, the patient moves lever 50 to the closed or dwell position. This further carries solution line 88 across base 12 of device 10. Eventually, ratchet 68 associated with cam 56 rotates counterclockwise across lock 26 locking lever 50 in the closed position. Here, cam 58 moves off of drain line 84 and cam 56 rotates counterclockwise to occlude patient line 82. During the closed position, the patient allows the fresh dialysate to dwell within the patient's peritoneum to remove waste, toxins and ultrafiltrate as is known.

After a dwell period, the patient removes lever 50 from base 12 by pulling pins 70 connected to hub 52 upward and through slots 24 in sidewalls 18 and 20. The patient discards the tubing set 80 and the emptied fresh dialysate bag and replaces these with another tubing set 80 and full solution bag. Upon doing so, the patient returns lever 50 to the drain position shown in FIGS. 1 and 11. The above described sequence is repeated a number of times according to the patient's prescribed therapy.

While lever 50 has been described herein as being operated manually, it is also expressly contemplated to couple hub 52 of lever 50 to a motor. For example, hub 52 could extend through one of sidewalls 18 or 20 and be supported in that sidewall by a set of ball or roller bearings. The motor can be a high precision motor, such as a stepper motor, that pivots the various ratchets 68 of cam 54 past lock 26 of base 12. The motor turns in the opposite direction such that its shaft becomes decoupled from hub 52, allowing tubing set 80 to pull lever arm 54 downwardly so that the current ratchet 68 of hub 52 is held against lock 26 of base 12. This sequence is repeated through each of the drain, flush, fill and dwell cycles. After dwell, the motor is decoupled from hub 52 such that lever 50 can be removed along with tubing set 80, so that the next tubing set can be reloaded. The motor returns the motor shaft to an initial position.

Alternatively, device 10 relies on the motor to hold hub 52 at a particular position, eliminating the need for ratchets 68 and lock 26. The motor can be operated manually, e.g., the patient pushes a pushbutton to cause a motor controller to energize the motor for the next action. Alternatively, the motor controller operates with a timer to rotate at preset times automatically. Here, the patient can sleep or otherwise concentrate on another activity during the drain, flush, fill and dwell cycles. Device 10 can also be provided with an alarm or beeper that makes or alerts the patient when the next solution bag and tubing set needs to be loaded.

### Dial Actuated Device

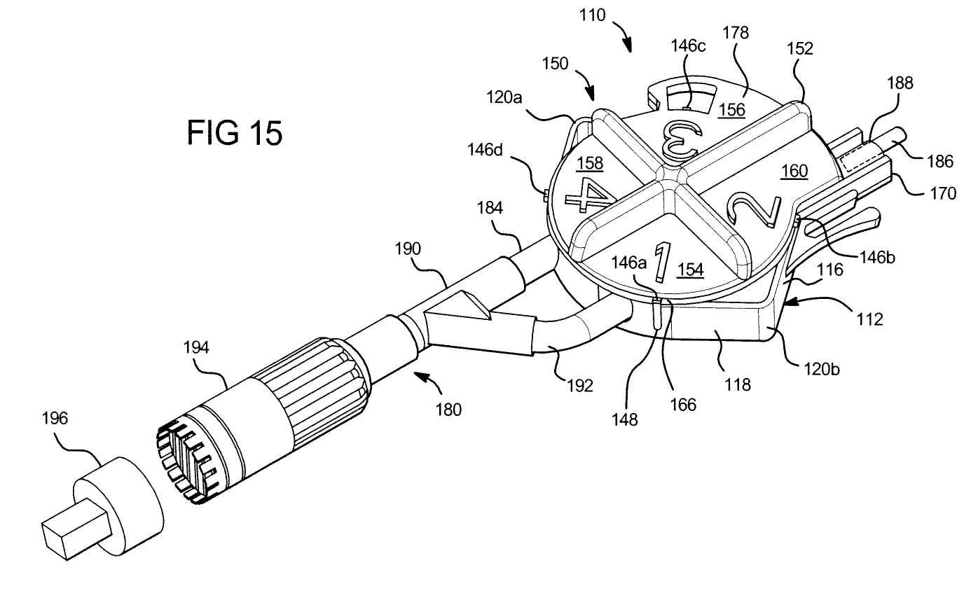

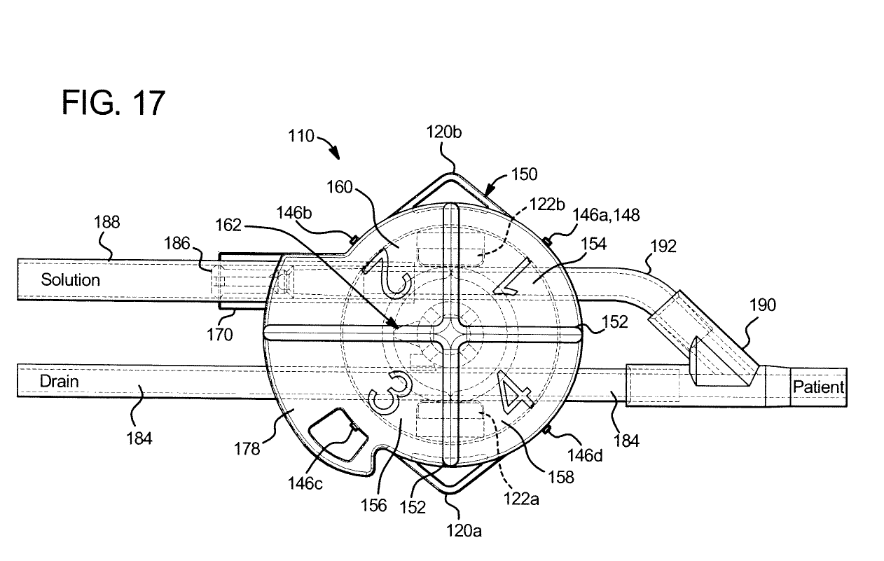

Referring now to FIGS. 15 to 19, a second primary embodiment for a flow control device for peritoneal dialysis is illustrated by device 110. Device 110 includes a base 112, a dial 150 rotatable with respect to base 112 and a tubing set 180, which is fitted operably into base 112, and which is acted upon by dial 150 as shown in detail below in connection with FIGS. 17 to 19. Base 112 and dial 150 are made of any suitable material, such as a plastic material. Dial 150 includes four visual alignment ribs 146a to 146d. Base 112 includes a visual alignment rib 148. Different dial ribs 146a to 146d align with base rib 148 as dial 150 is rotated about base 112 as seen in detail below.

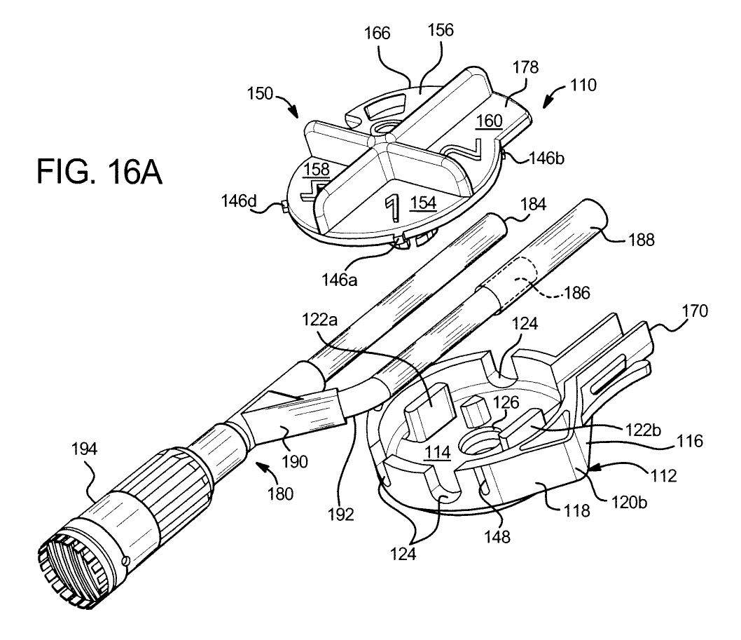

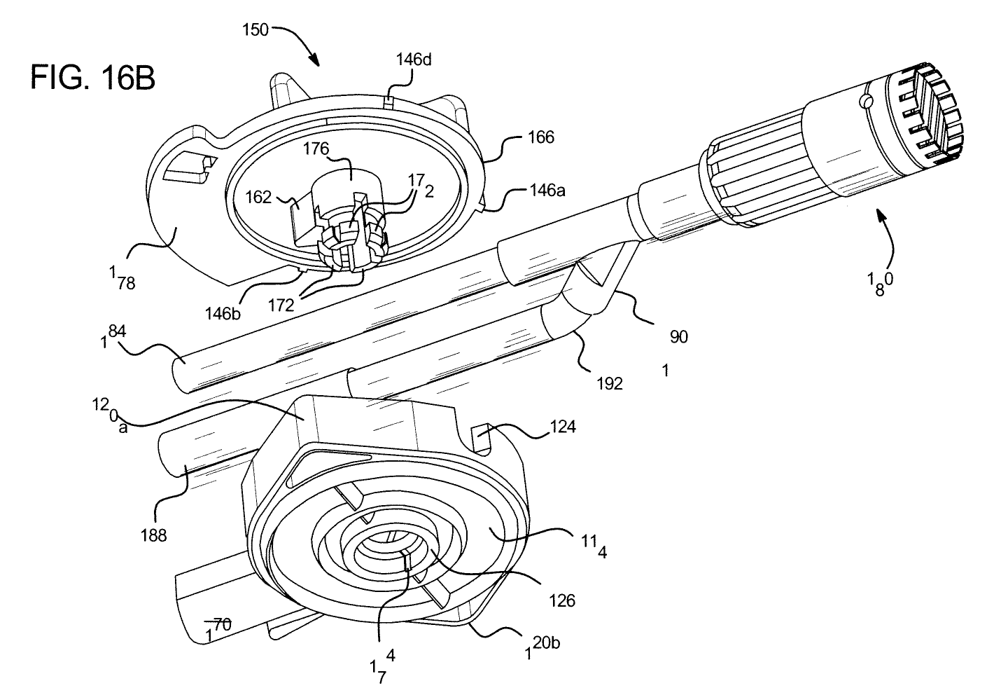

Base 112 includes a bottom 114 (seen best in connection with FIGS. 16A and 16B). Sidewalls 116 and 118 create points 120a and 120b that fit the user's hand in an ergonomic fashion. The indicators for position are the alignment ribs, one located on the base and four located on the dial, one at each position, e.g., arrow in FIG. 17 that points to the alignment/position ribs noted as “Position 1”.

FIGS. 16A and 16B to 19 illustrate that at least one pressure plate 122a and 122b extends upwardly from bottom 114. As seen in FIGS. 17 to 19, tubes of tubing set 180 reside along an inner surface of pressure plates 122a and 122b. Pressure plates 122a and 122b accordingly perform an alignment function for enabling tubing set 180 to be readily and snuggly positioned within base 112. As seen in FIG. 16A, sidewalls 116 and 118 define openings 124 that enable tubing set 180 to be placed flush against bottom 114, and so that dial 150 can be mounted onto base 112 without crimping any of the tubes of tubing set 180.

Base 112 defines an opening or aperture 126 that accepts a snap-fitting pin 164 shown in phantom in FIGS. 17 to 19. Pin 164 extends downwardly in one direction from a wall 166 of lid 150. Snap-fitting pin 164 snap-fits through aperture 126 for easy assembly and in such a manner that dial 150 can be rotated with respect to base 112.

A handle or twisting mechanism 152 extends upwardly from wall 166 of dial 150. Handle 152 as illustrated also divides dial 150 into four quadrants, namely a drain quadrant 154, a flush quadrant 156, a fill quadrant 158 and a break frangible quadrant 160. Each of the quadrants is marked with identifying indicia, such as “drain,” “flush,” “fill,” and “break frangible.”

Tubing set 180 as illustrated includes a patient line (not seen here), a drain line 184, a solution line 188 and a Y-connector 190, which enables drain line 184 and patient line 182 to communicate fluidly with solution line 188. FIG. 16A illustrates that a separate tubing section 192 connects to Y-connector 190 and solution line 188. Solution line 188 also houses frangible seal 186 as discussed above (for one, e.g., European setup, North American setup places frangible at the solution bag). Each of the patient line 182, drain line 184 and solution line 188/192 is connected sealingly to Y-connector 190 as seen in FIGS. 15, 16A and 16B.

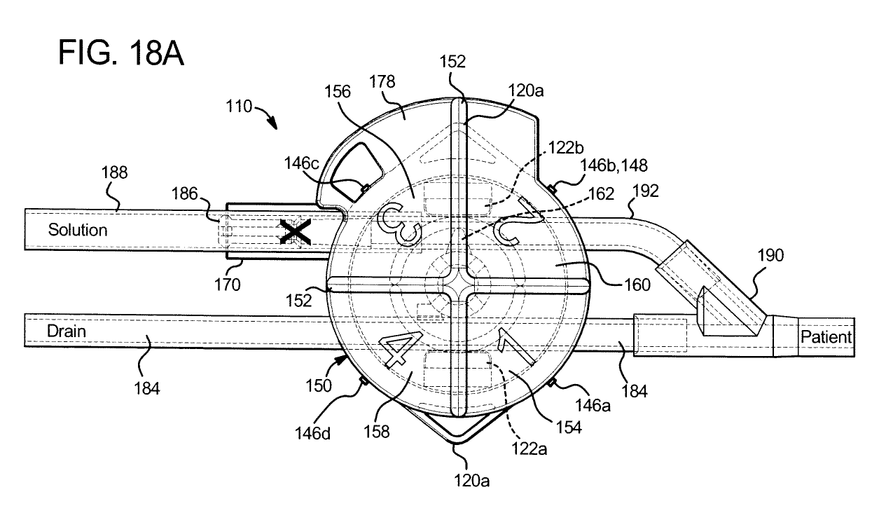

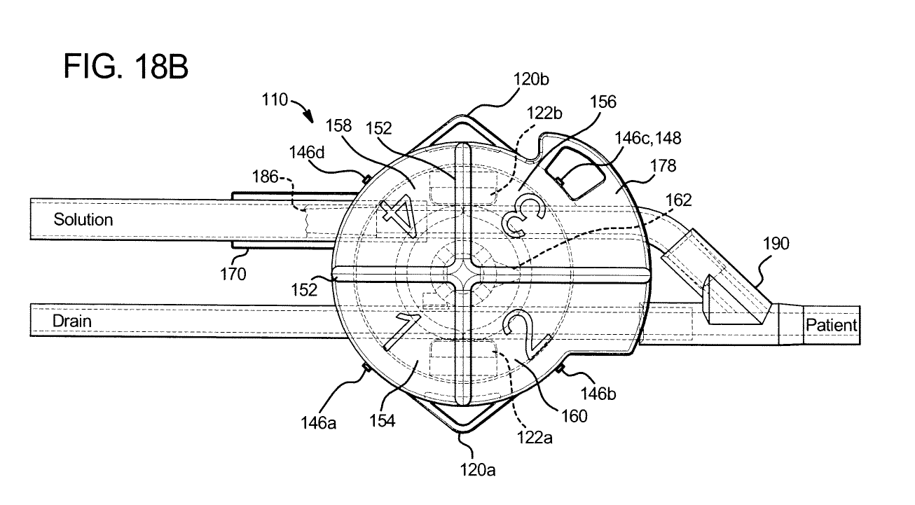

Dial actuated device 110 in one embodiment is supplied to the patient already assembled, in the drain position and ready to use as indicated in FIG. 17 “as received by patient”. In FIG. 17, the frangible seal 186 cannot be broken. Extension 170 from base 112 is configured to prevent breaking of frangible seal 186 in a downward fashion against the base 112. Extension 170 cradles the frangible seal 186 so that a downward pull (from the perspective of the drawing) will not break frangible 186. Flange geometry 178 is added to dial 150, so that in the drain position frangible seal 186 cannot be broken by an upward pull (from the perspective of the drawing). When device 110 is in the “break frangible” position in FIG. 18A, flange geometry 178 is rotated out of the way, allowing for frangible seal 186 to be broken in the upward direction (from the perspective of the drawing).

Y-connector 190 is connected in turn to a transfer set connector 194. A cap 196 is pulled from a transfer set connector 194. Transfer set connector 194 then threads onto a mating connector of the transfer set connected to the patient. In one embodiment, connection between connector 194 and the patient's transfer set is done aseptically. One suitable connector is disclosed in connection with U.S. patent application Ser. No. 10/074,532, entitled Dialysis Connector and Cap Having an Integral Disinfectant, filed Feb. 11, 2002, assigned to the assignee of the present disclosure, the entire contents of which are hereby incorporated by reference.

FIG. 16B illustrates that the bottom side of wall 166 of dial 150 includes locking, e.g., angled projections 172 that lock releasably and sequentially into a mating groove 174 as dial 150 is rotated from position to position. Projections 172 and mating groove 174 hold device 110 in a desired state until dial is moved. Projections 172 and mating groove 174 also provide audible and tactile feedback to the user that the device has reached the next state.

Referring now to FIGS. 17, 18A, 18B and 19, one method for operating dial actuated flow control device 110 is illustrated. In FIG. 17, dial 150 is initially in a “drain” position, which is the position shown in FIGS. 15 and 16A, and wherein alignment rib 146a (#1) of dial 150 is aligned with alignment rib 148 of base 112. Here, an occluder or pawl 162 occludes no tube. Pawl or occluder 162 can depend from wall 166 and/or be attached to detent collar 176 as seen in FIG. 16B. Pawl or occluder 162 includes a rounded point to reduces the torque needed to occlude the lines fully.

Frangible 186 blocks solution line 188 and spent or effluent dialysate is allowed to flow from the patient, through Y-connector 190 and through drain line 184 to drain. The patient is therefore able to drain spent dialysate from the patient's peritoneum, through the patient's transfer set, connector 194, Y-connector 190, patient line 182, and through drain line 184 to drain. The spent fluid will also flow through the other leg of Y-connector 190, through extension 192 and against frangible seal 186. This remaining spent solution is then flushed from tubing set 180 prior to the patient being filled with fresh dialysate, as shown in connection with FIG. 18B.

In FIG. 18A, the patient or user grasps handle 152 and rotates dial 150 clockwise ninety degrees, so that the alignment rib 146b (#2) of lid 150 is aligned with alignment rib 148 of base 112. Device 110 is now in a break frangible condition. Here, occluder or pawl 162 occludes solution tube so that when the patient breaks frangible 186 (act shown by darkened “X”, fresh solution cannot flow through device 110 until the patient is ready for the fresh solution to flow. Flange geometry 178 is rotated out of the way exposing the frangible 186 for breakage in an upward manner (with respect to illustrated positioning of device 110). Extension 170 of base 112 protects frangible 186 from premature breakage in a downward and/or side-to-side manner (with respect to illustrated positioning of device 110).

FIG. 18B illustrates a flush cycle using connector 110. Because Y-connector 190 resides outside flow control device 110, flow control device 110 does not have the capability of occluding tubing set 180 on the patient side of the Y-connection. Accordingly, in this embodiment the patient clamps the patient side of connector 190 just prior to performing the flush cycle with an external clamp (not illustrated) known to those of skill in the art. The patient transfer set has an incorporated twist clamp, which allows the patient to shut off the flow to the patient's peritoneum independent of dial device 110.

In FIG. 18B, the patient or user grasps handle 152 and rotates dial 150 clockwise another ninety degrees, so that the alignment rib 146c (#3) of lid 150 is aligned with alignment rib 148 of base 112. Device 110 is now in a flush condition. Here, occluder or pawl 162 occludes no tube. Frangible 186 is broken as seen in FIG. 18B. Now, fresh solution can flow through solution line 188, including extension 192, through connector 190 and into patient line 182 and drain line 184. The flush is performed for a period of time suitable to rinse spent dialysate from the transfer set/Y-connector 190 area of tubing set 180. The flush also helps to wash away any bacteria that may have been introduced into the fluid system via the connection of transfer set connector 194 to the transfer set.

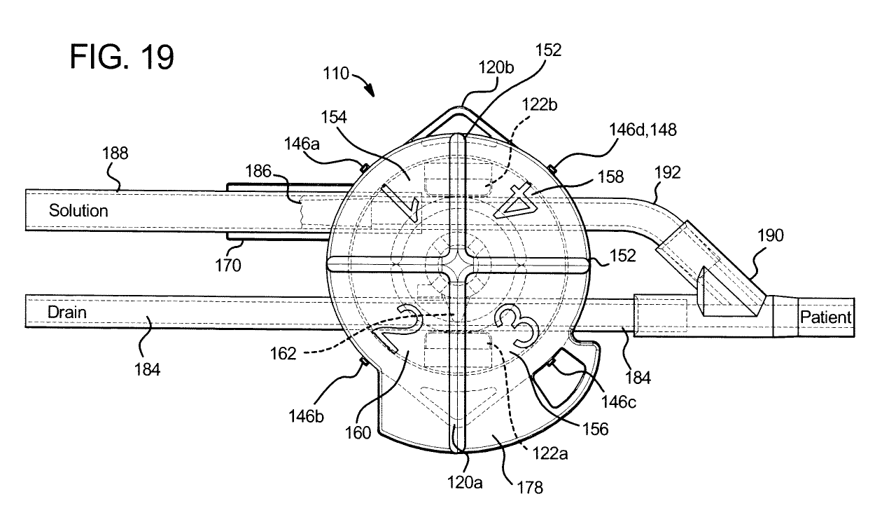

Referring now to FIG. 19, a fill cycle using dial control device 110 is illustrated. Here, the patient or user twists dial 150 using handle 152 clockwise again ninety degrees, so that the alignment rib 146d (#4) of lid 150 is aligned with alignment rib 148 of base 112. This action causes occluder or pawl 162 to contact and occlude drain line 184. Frangible 186 is broken as seen in FIG. 18B. Fresh solution can therefore flow from solution line 188 through connector 190 into the transfer set but is not allowed to flow additionally through drain line 184 to drain. The tubing configuration of FIG. 19 allows the fill bag to be completely emptied to the patient. The patient then closes the transfer set. Preassembled device 110 is discarded.

In FIGS. 17, 18A, 18B and 19, feedback producing and state holding devices 172 discussed above lock into mating groove 174 (FIG. 16B) when a state is reached. Devices 172 and mating groove 174 hold device 110 in the set position until it is once more rotated clockwise ninety degrees.

### Twist Activated Device

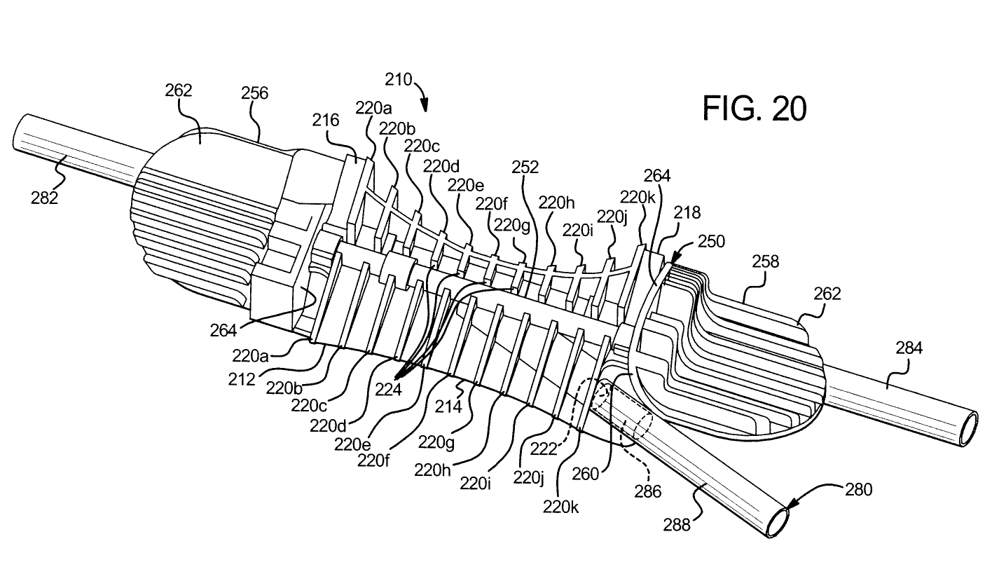

Referring now to FIGS. 20 to 24, a third primary embodiment for a flow control device is illustrated by twist activated device 210. Device 210 includes a base 212 and a twistable clamp 250, which is twistable with respect to base 212 to kink tubes 282, 284 and 288 of tubing set 280 desirable to kink one or more of patient line 282, drain line 284 and/or solution line 288. Base 212 and twistable clamp 250 can be made of any suitable material, such as polypropylene, polysulfone or polycarbonate.

Base 212 includes a bottom wall 214 that extends to a first end 216 and a second end 218. Ribbed pairs 220a to 220k extend up from bottom wall 214. Ribbed pairs 220a to 220k perform multiple functions. One function is to define apertures 222 for holding and directing patient tube 282, drain tube 284 and solution tube 288 within device 210. Apertures 222 are angled to allow proper flow of fluid from solution tube 288 to the other of the first and second tubes. A second purpose is to provide opening 224 to hold a rod 252 of twist clamp 250.

Rod 252 in an embodiment is snap-fitted into openings 224 defined by ribs 220 (referring collectively to ribs 220a through ribs 220k). To aid in the snap-fitting arrangement, rod 252 in an embodiment includes at least one expanded section 254.

Twist clamp 250 includes first twistable occluding member 256 and second twistable occluding member 258. In one embodiment, rod 252 is held fixed and not rotatable within openings 224 defined by ribs 220. Here, each of occluders 256 and 258 is fitted rotatably onto the ends of rod 252, so that each occluder may be twisted independently with respect to rod 252. In an alternative embodiment, rod 252 is rotatably engaged within apertures 224 of ribs 220. Here, one of occluders 256 or 258 is rotatable with respect to rod 252, while the other occluder 256 or 258 is fixed and not rotatable with respect to rod 252, that is, it rotates with rod 252.

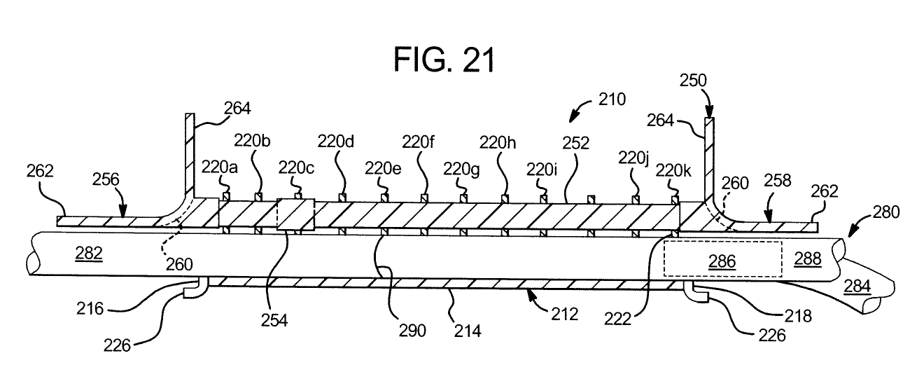

In any case, occluders 256 and 258 are intended to be twistable or rotatable independently of one another to create a desired crimping pattern for tubes 282, 284 and 288 of tubing set 280. As seen in FIGS. 21 to 24, solution tube 288 and drain tube 284 come into fluid communication with patient line 282 at Y-connector 290. Y-connector 290 is maintained within flow control device 210, similar to the configuration of flow control device 10, and different from the configuration of flow control device 110. The configuration here enables patient line 282 to be occluded in addition to the occlusion of drain line 284 and solution line 288.

Each of occluders 256 and 258 includes a variable crimping resistance cam engaging surface 260, which extends from a low resistance point at the interface with patient twist tab 262, radially upwardly to a flow occluding or crimping surface 264. Bottom wall 214 of base 212 further includes tactile feedback producing tips 226 positioned (e.g., two tips 226 along the outsides of each of edges 216 and 218) to provide engaging tactile feedback to the patient when twistable occluders have been twisted in one direction or another fully to occlude or crimp one of tubes of tubing set 280.

FIG. 21 illustrates a drain sequence in which the patient does not twist either of occluding members 256 or 258. Here, frangible seal 286 remains intact within solution line 288 so that no spent fluid from the patient is able to flow past frangible seal 286 (for one, e.g., European setup, North American setup places frangible at the solution bag). Instead, spent fluid flows from the patient's peritoneum, through the transfer set, through patient line 282, and through drain line 284, to drain.

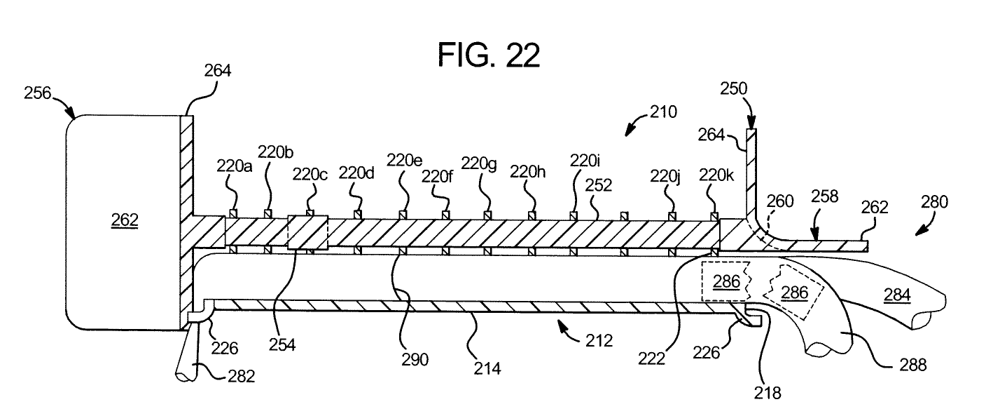

When the drain cycle is complete, the patient performs a flush cycle using twist device 210 as seen in FIG. 22. Here, the patient turns left occluder 256 in a counterclockwise direction until the patient feels tactile feedback from feedback device 226. At this point, patient line occluder 286 has crimped patient line 282 completely. The patient does not move right occluder 258. However, the patient breaks frangible seal 286 within solution line 288 by pulling solution line 288 over end 218 of base 212 causing seal 286 to crack open. This allows fresh fluid to flow through solution line 288, through connector 290, and out drain line 284 to flush old fluid and any bacteria through the Y-connector and drain line 284 to drain.

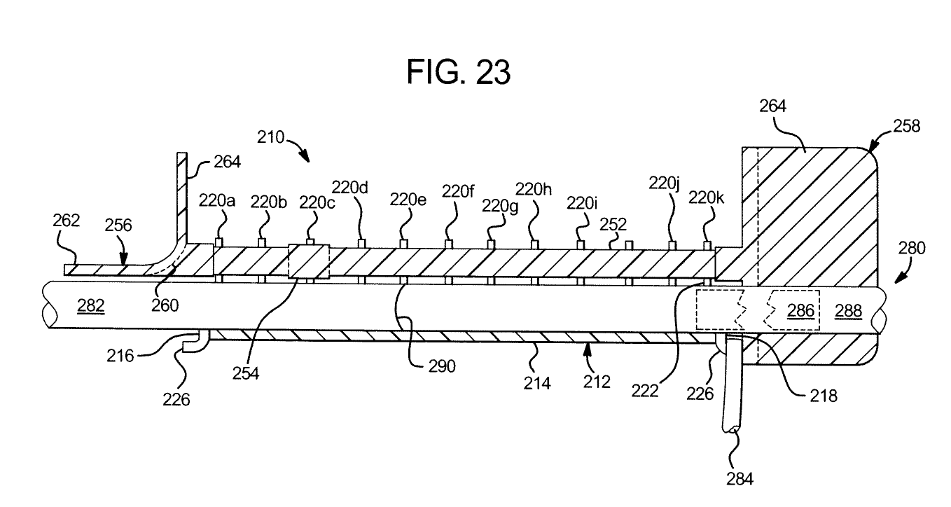

Referring now to FIG. 23, a patient fill cycle using twist device 210 is illustrated. Here, the patient turns right occluder 258 as illustrated to crimp or close off drain line 284. When the patient turns occluder 258 an appropriate distance, tactile feedback via feedback producing device 226 is provided so that the patient knows not to turn right occluder 258 any further. The patient then turns the patient line occluder 256 in the appropriate direction back to its original position of FIG. 21 so that patient line 282 is now open. Now, drain line 284 is occluded allowing fresh solution to flow from solution line 288 past broken occluder 286, through Y-connector 290 and patient line 282, through the patient's transfer set and into the patient's peritoneum. The configuration of FIG. 23 is maintained until all fresh solution has been drained from the solution bag and allowed to flow into the patient.

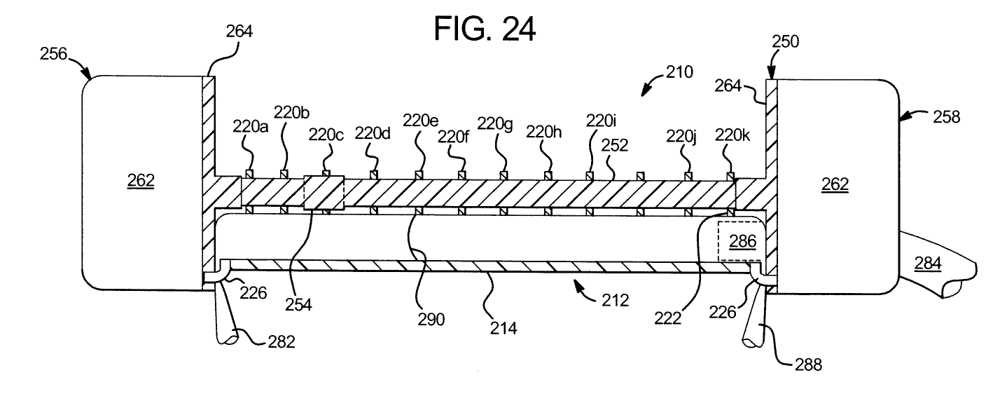

Referring now to FIG. 24, a dwell configuration for device 210 is illustrated. The dwell configuration is analogous to the close or dwell configuration of lever arm device 10 shown in FIG. 14. Here, patient line occluder 256 is turned in the clockwise direction a distance sufficient to occlude patient line 286 as communicated to the patient via tactile feedback device 226. The patient also turns right occluder 258 to the position shown in FIG. 24 to occlude solution line 288. This isolates the patient from any air remaining in the solution bag as much as possible. After dwell period is complete, the patient disconnects from the patient connector (not shown) and drains the drain bag.

In an alternative embodiment, occluder 258 in the drain cycle is turned to crimp solution line 288, allowing spent fluid to flow from the patient, through the transfer set, patient line 282, Y-connector 290 and drain line 284 to drain. This configuration eliminates the need for frangible seal 286. To flush, the patient turns occluder 256 to crimp patient line 282 and turns occluder 258 to open solution line 288 as seen in FIG. 22. Fill and Dwell cycles are then performed according to FIGS. 23 and 24.

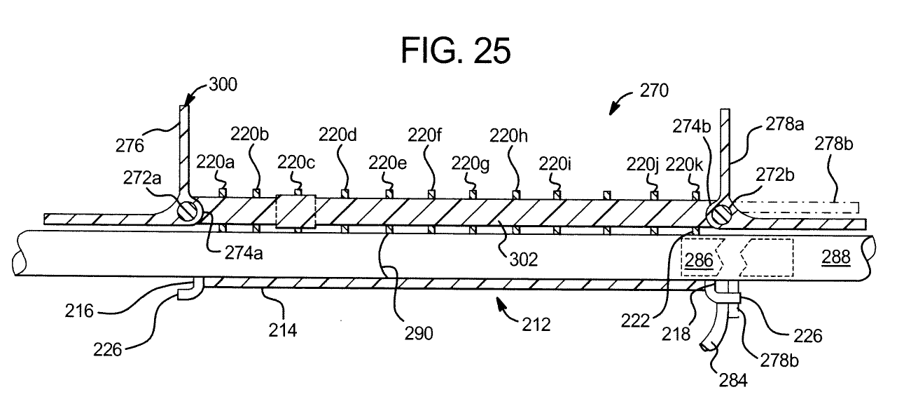

Referring now to FIG. 25, an alternative flow control device 270, which is similar to that of flow control device 210 is illustrated. Here, rotating or twisting occluders 256 and 258 are replaced by hinged occluders 276 and 278a and 278b. Rod 302 of crimping occluder 300 is fixed with respect to base 212 of flow control device 270. Alternatively, rod 302 is formed integrally with ribs 220a to 220k of base 212.

Rod 302 defines or includes hinge pins 272a and 272b at either end. Pins 272a and 272b are fitted into cylindrical bores 274a and 274b formed in occluders 276 and 278a/278b, respectively. Locking and feedback mechanisms 226 lock the occluders in the downward crimping position and provide tactile feedback to the patient or user that the respective occluder is in the full occluding position. Ratcheting mechanisms (not illustrated), similar to those of lever device 10 may be used alternatively or additionally.

The occlusion steps for the drain, flush, fill and dwell cycles are the same as set forth above in connection with FIGS. 21 to 24 of device 210. FIG. 25 shows a fill cycle to illustrate that occluders 278a and 278b are split from one another so as to operate independently to occlude the drain line 284 in the fill cycle and alternatively to occlude solution line 288 in the dwell cycle (or both drain line 284 and solution line 288 are occluded during the dwell cycle.

Occluders 278a and 278b can again eliminate the need for frangible seal 286. If so, occluder 278a is hinged downward to crimp solution line 288 in the drain cycle. When drain is complete, occluder 278a is lifted to allowed flow from solution bag through solution line 288 in the drain cycle, while single patient occluder 256 is flipped down to occlude patient line 282 for flush. As before, base 212 includes tactile feedback apparatuses 226 at each end 216 and 218 of base 212, which tell the patient when occluder 276 or 278a/b is in proper position and in an embodiment to releasably lock the appropriate occluder in place.

In the fill cycle of FIG. 25, occluder 278b is flipped down to occlude drain line 284, while occluders 276 and 278a are left up to allow fresh solution to flow from the solution bag to the patient. In the dwell cycle, all three occluders can be flipped down to occlude all three patient, drain and fill lines in one embodiment. This isolates the patient from the remainder of the disposable set as much as possible.

### Three Piece Dial Flow Control Device

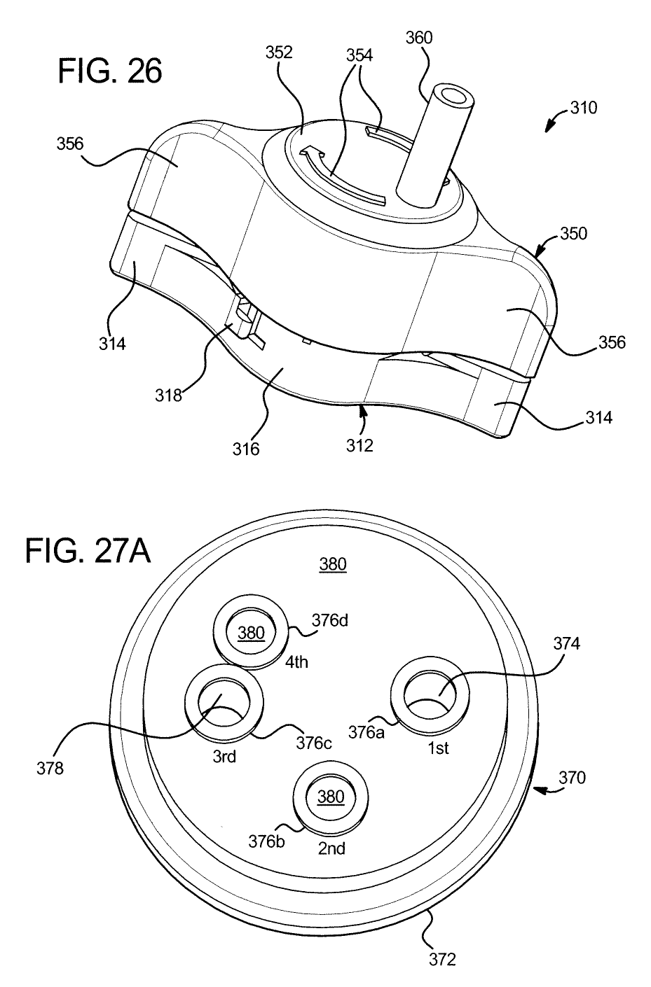

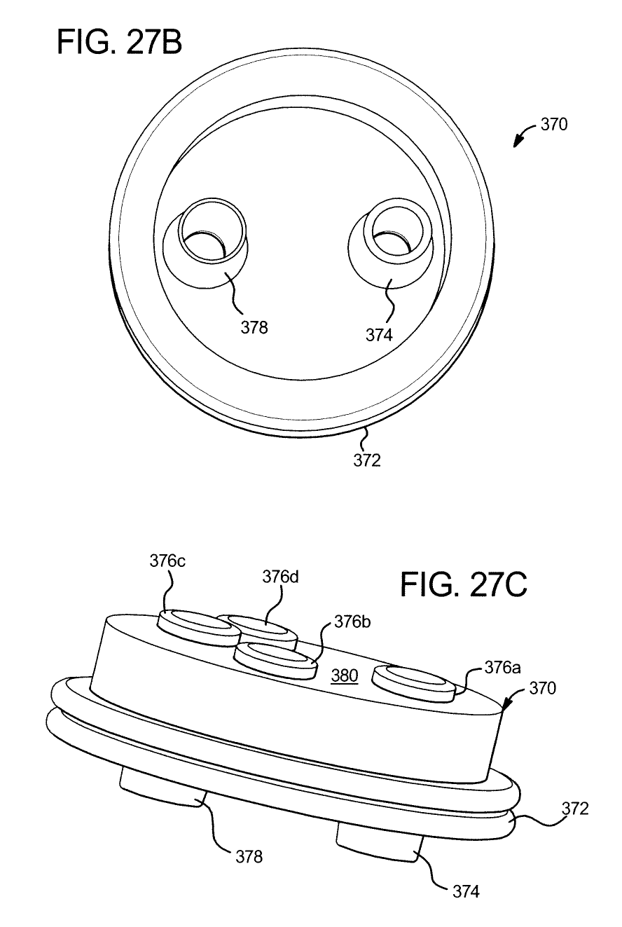

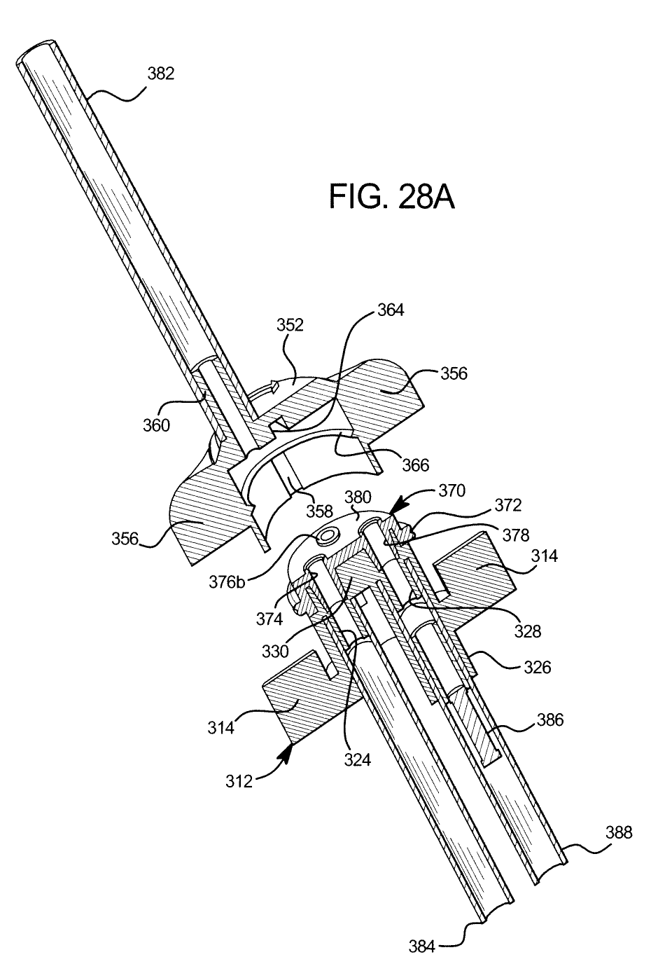

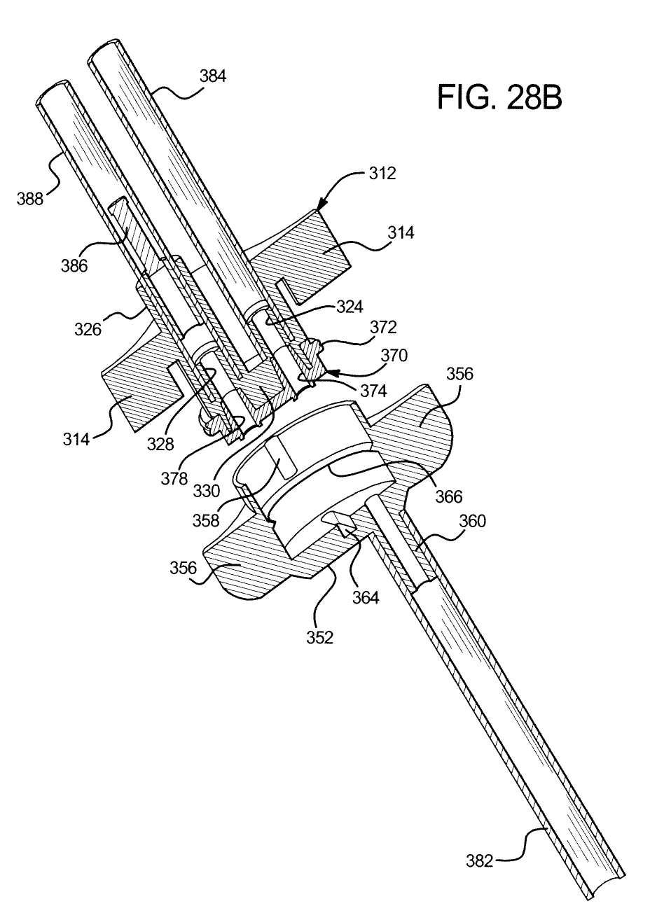

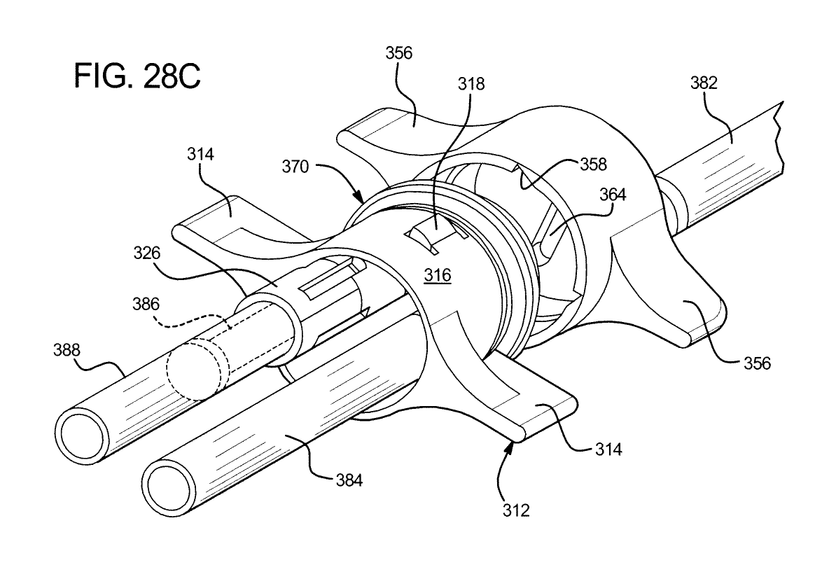

Referring now to FIGS. 26, 27A to 27C, 28A to 28C, 29, 30, 31A, 31B, 32A to 32E, 33A to 33E and 34A to 34F, a fourth primary embodiment for a flow control device is illustrated by device 310. FIG. 26 shows device 310 as assembled, which includes a single or patient line cap 350, which is connected rotatably to a dual or solution/drain line cap 312. FIGS. 27A to 27C show a flexible gasket 370, which is fitted onto two line cap 312, as seen for example in FIGS. 28A to 28C. Caps 312 and 350 eliminate the Y-connection between the patient, solution and drain lines shown above.

In an embodiment, caps 312 and 350 are made of a suitable medical grade polymer, such as polycarbonate or polysulfone or any of the other materials listed herein. Gasket 370 is made of a suitable flexible and seal making material, such as silicone or isoprene rubber.

Single line cap 350 includes or defines a top 352, which is circular in the illustrated embodiment. Top 352 includes or provides arrows or indicators 354, which inform the patient as to the direction to turn single line cap 350 relative to dual line cap 312. To facilitate the relative twisting, single line cap 350 includes or defines tabs or twisting apparatuses 356, while dual line cap 312 includes or defines tabs or twisting apparatuses 314. Twisting apparatuses 356 and 314 enable a patient to turn cap 350 relative to cap 312 readily and without having to apply an undue amount of torque.

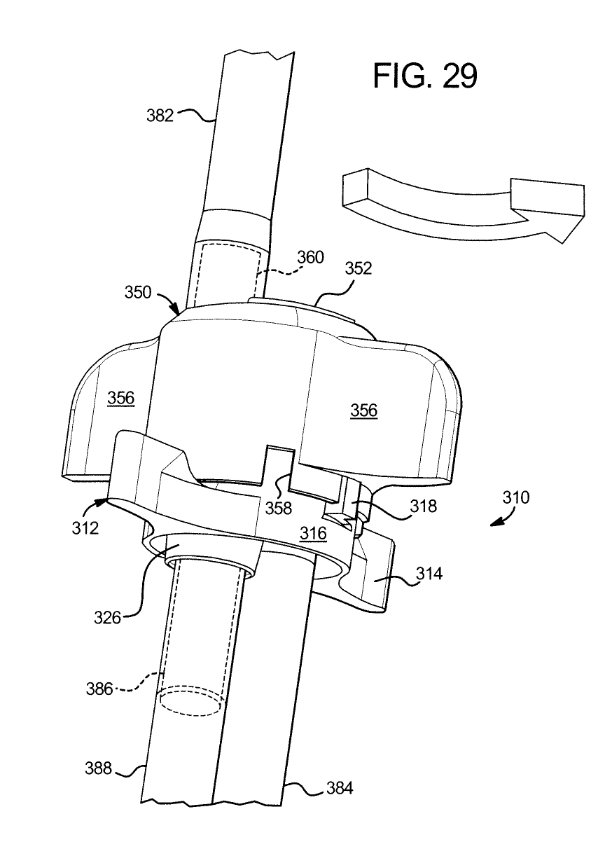

Two line cap 312 includes or defines a cylindrical hub 316 having an anti-reverse and overtravel tab 318, as seen in FIGS. 26, 28A to 28C and 29. Overtravel and anti-reverse tab 318 is spring-like, e.g., fixed at one end but free to flex at the other end, and locks into grooves 358 (FIGS. 28A to 28C) or notches 358 (FIG. 29) defined by single line cap 350. These apparatuses can provide audible and/or tactile feedback indicating that a next flow state has been reached or established. The tab and groove engagement locks cap 350 removably in a desired position relative to two line cap 312, preventing cap 350 from being turned too far past a desired position and from traveling backwards from a desired position. Tab 318 is somewhat flexible and biased to allow the patient to twist cap 350 from a locked position in the correct direction to a second desired and locked position. Tab 318 is not flexible or as flexible in the reverse direction, so that the patient cannot, at least without providing undue torque, turn twisting cap 350 in a reverse and undesired direction. The engagement of tab 318 and grooves 358 or notches 358 also provides tactile and possibly audible feedback to the patient, so that the patient knows when the next therapy step or valve state has been achieved.

As seen best in FIGS. 26, 28A to 28C, 29 and 30, single line cap 350 includes or defines a port 360, which in the illustrated embodiment connects sealingly to patient line 382. Port 360 extends outwardly from the top 352 of cap 350. Cylindrical hub 316 as seen in FIGS. 28A, 28B and 29 is hollow underneath and defines inset ports 324 and 328, which accept and connect sealingly to drain line 384 and solution line 388, respectively. As seen best in FIGS. 28A to 28C and 29, dual line cap 312 also includes or defines a cylindrical well 326 into which solution line 388 is inserted. Cylindrical well 326 provides a rigid, sturdy surface against which solution line 388 can be bent to break frangible seal 386 to allow fresh solution to flow through cap 312, into single line cap 350, to a desired destination, e.g., for flush or fill (frangible seal 386 provided at device 310 only in European version, North American version does not have frangible seal 386 provided at device 310 as discussed below).

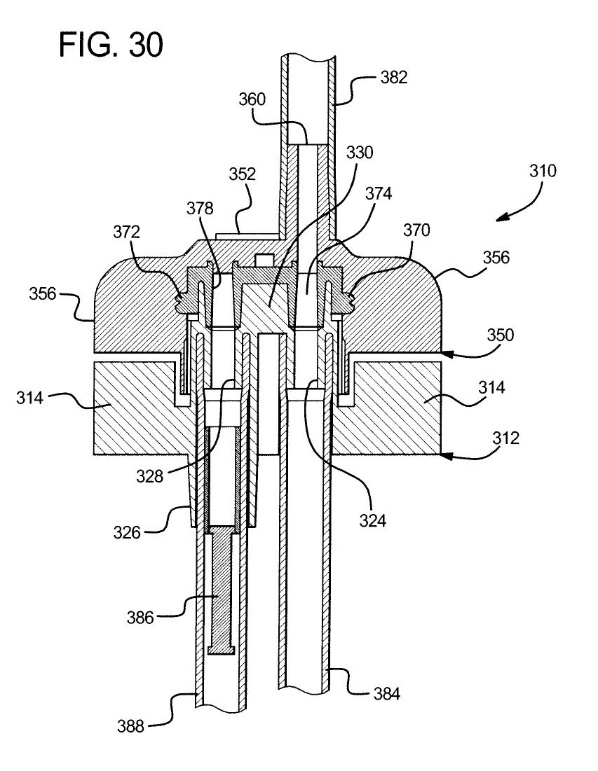

As seen best in FIGS. 28A, 28B and 30, cylindrical hub 316 of dual line cap 312 includes or defines a head 330, which includes an inner section and an outer angular ring 332. The inner section and outer ring 332 of head 330 fit sealingly into the mating contour of flexible seal 370. Seal 370 includes or defines nipples 374 and 378 that mate with drain line port 324 and solution line port 328 of dual line cap 312, respectively. In this manner, fluid in drain line 384 and solution line 388 communicates with nipples 374 and 378 of flexible seal 370.

As seen in FIGS. 27A to 27C, gasket 370 defines a double-lipped seal 372, which seals around inner annular raised rib 366 (e.g., FIGS. 28A and 28B) of patient line cap 350 to provide a sealed and rotational engagement between patient line cap 350 and gasket 370. Gasket 370 is fitted sealingly onto two line cap 312. Seal 372 accordingly enables device 310 to have a sealed and rotatable operation.

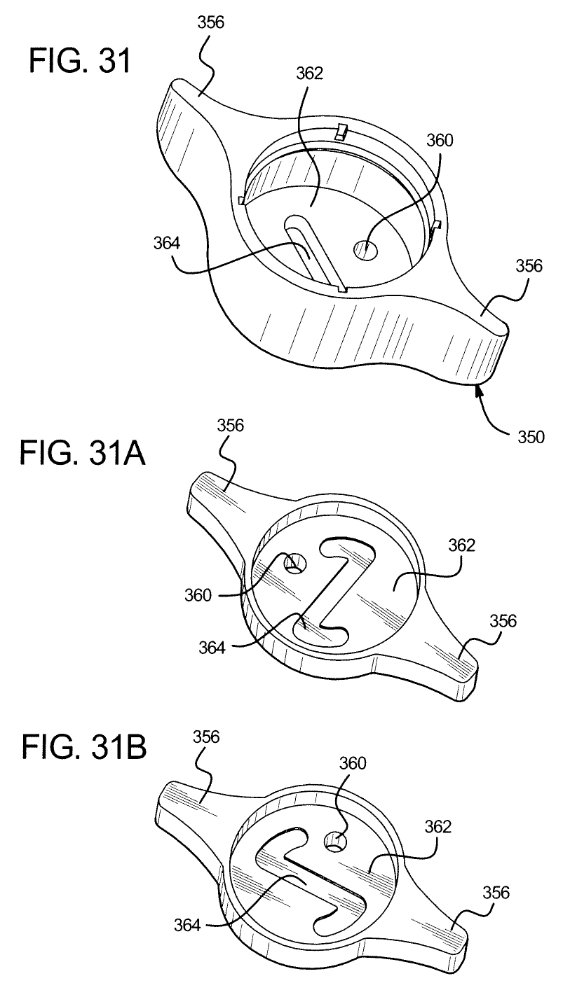

FIGS. 27A to 27C show that drain line nipple 374 and solution line nipple 378 each open to surface 380 surrounded by a raised or volcano referring to seal 376a and 376c, respectively. Raised or volcano seals 376 seals (referring collectively to seals 376a to 376d) seal against an inner surface 362 of top 352 of single line cap 350 as seen best in FIGS. 28A, 28B, 31A and 31B. Raised or volcano seals 376 are defined or placed onto top surface 380 of flexible seal 370 as seen in FIGS. 27A to 27C either around holes or in desired areas to create blind seals. Surface 380, that is resides inside and outside of blind seals 376b and 376d. Blind seals 376b and 376d are placed at positions to which patient line 382 connected to port 360 of single line cap 350 is rotated during therapy.

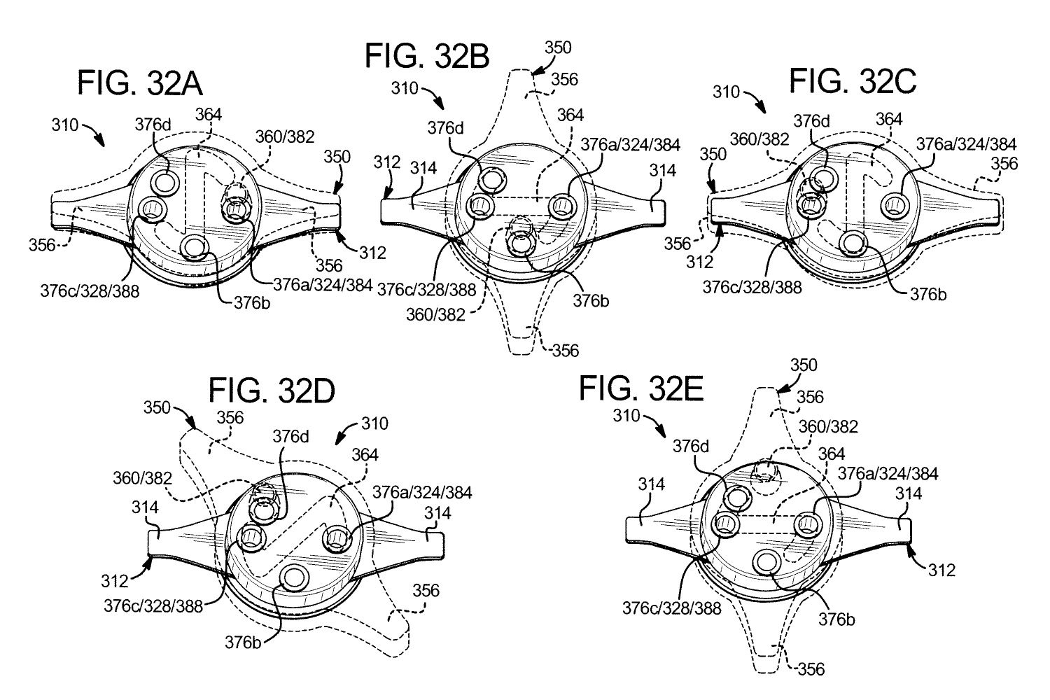

Slot 364 is used for example to produce an all-lines-open step as seen in FIGS. 32E, 33E and 34F to allow patient line 382 connected to cap 350 to communicate fluidly with a drain bag for example. Blind slot 364 in the all-lines-open state carries any remaining fluid from the supply and drain bags, through patient line 382, to drain. Blind slot 364 in the embodiments illustrated herein has a Z-shape. The shape of blind slot 364 of patient line cap 350 can be modified as necessary to produce a desired flow sequence as patient line cap 350 is turned relative to dual line cap 312.

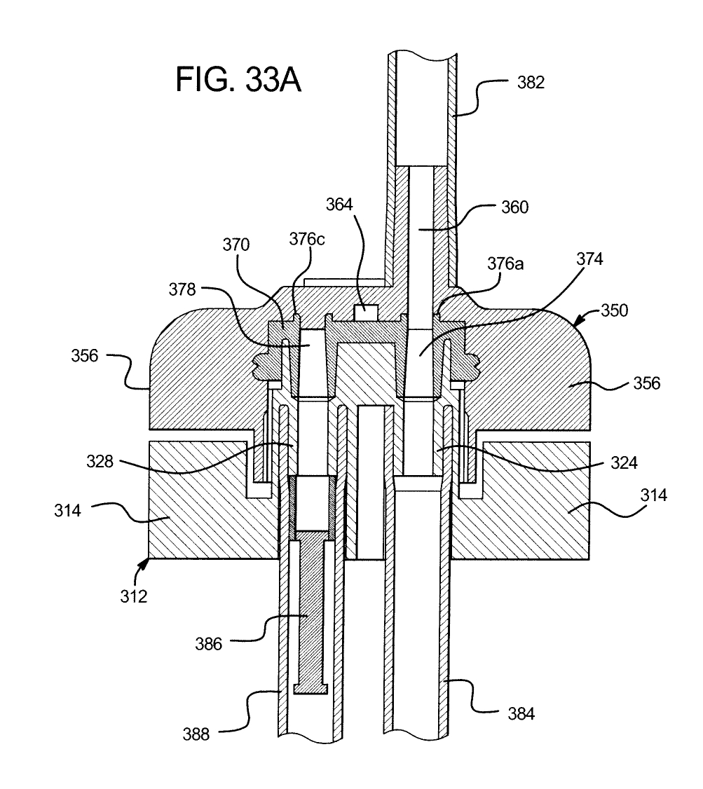

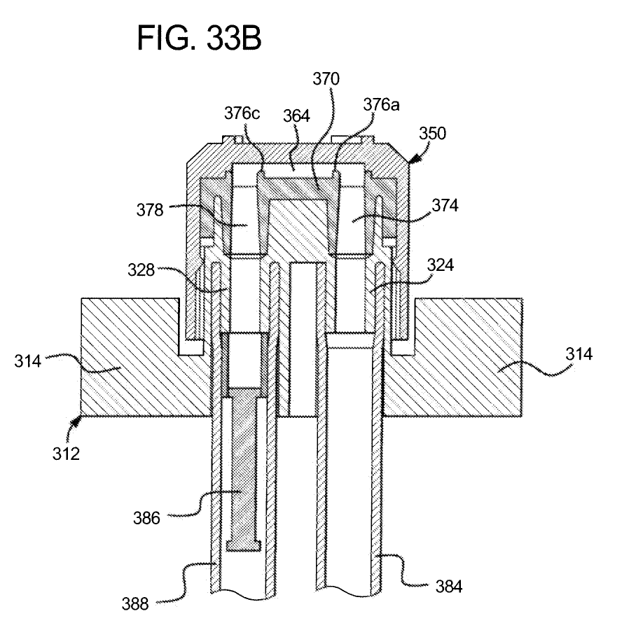

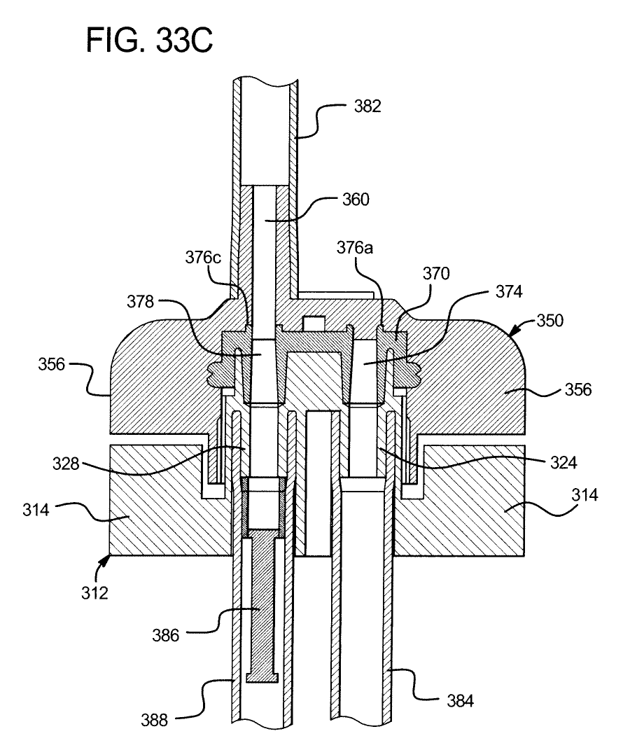

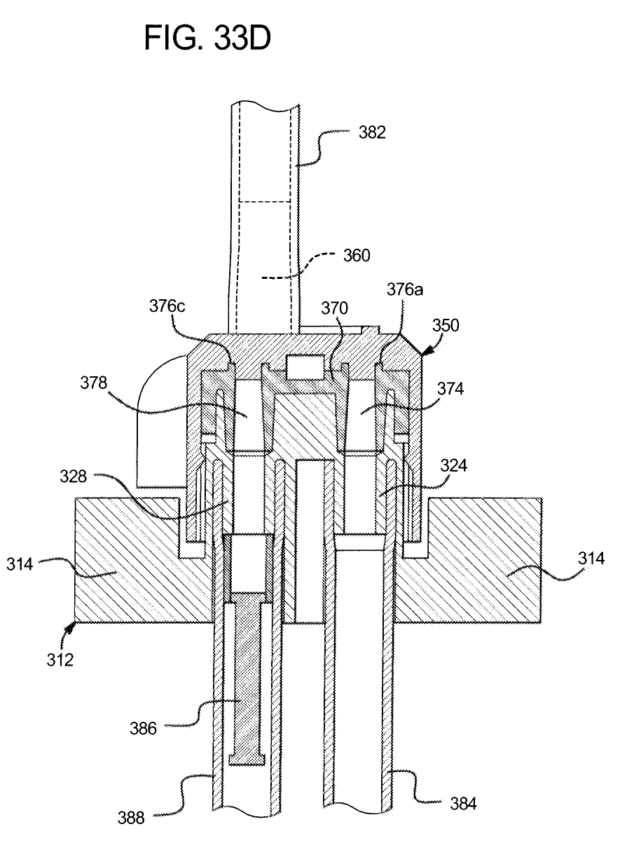

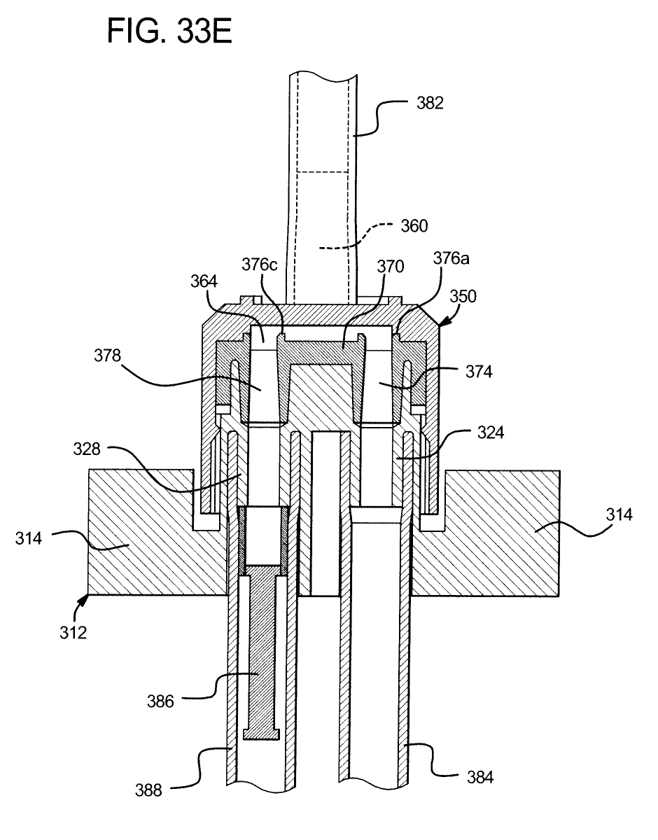

For purposes of explaining the fluid flow paths of FIGS. 32A to 32E and corresponding FIGS. 33A to 33E, it is helpful to think of patient line port 360 of cap 350 and corresponding patient line 382 rotating in steps to the different locations marked on diaphragm 370 of FIG. 27A. In a first step (for European treatment as will become clear below) viewing FIG. 27A, patient line 382 resides above drain nipple 374 of gasket 370 to allow the patient to drain spent solution. Here, raised seal 376a seals around the lumen patient line port 360 at the underside 362 of cap 350. In a second step, patient line port 360 and patient line 382 are rotated over blind seal 376b, for a flush and prime step in which patient line 382 is closed. When frangible 386 is broken, fresh solution flows in this second position through the drain line to flush same. In a third or fill step, patient line port 360 and corresponding patient line 382 are rotated over sealed solution line nipple 378 and raised seal 376c of gasket 370. Here, fresh solution flows through patient line 382 to the patient. In the fourth step, solution and drain lines are open with the patient line closed, the patient line being rotated over blind seal 376d at the fourth position viewing FIG. 27A. In a final all-lines-open position, patient line 382 and port 360 are rotated away from blind seals 376b and 376d. Blind slot 364 of cap 350 is rotated into communication with drain line 384 and solution line 388, which allows fluid to travel under wall 362 of cap 350 from the drain and solution lines, through slot 364 of cap 350 and into patient line 382 to a house drain or drain container.

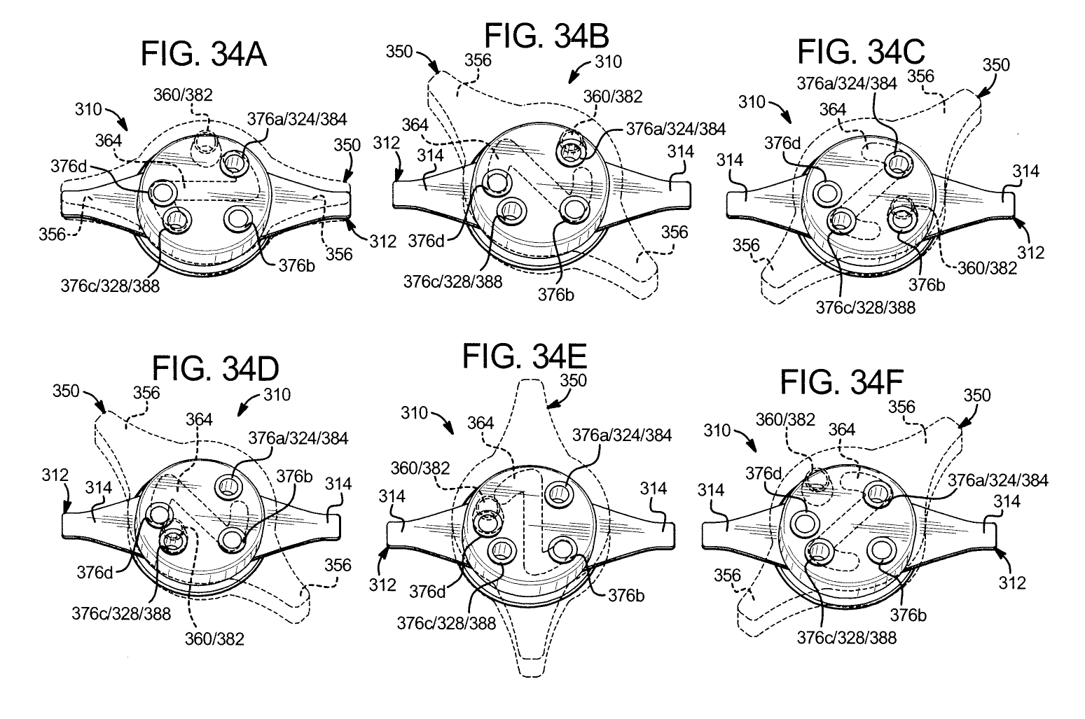

Referring now to the top views of FIGS. 32A to 32E and corresponding side views of FIGS. 33A to 33E, the above-described sequence is illustrated in further detail. FIG. 32 (referring collectively to FIGS. 32A to 32E) and FIG. 33 (referring collectively to FIGS. 33A to 33E) show one possible sequence, which is typical for a European CAPD therapy. FIG. 34 (referring collectively to FIGS. 34A to 34F) show a six-step sequence, which includes an initial vent step as seen in connection with FIG. 34A, which is typical for a U.S. CAPD therapy.

As seen in FIGS. 32A and 33A, in a first European step a patient drain is performed. Here, patient port 360 and patient line 382 of cap 350 are aligned with raised seal 376a of gasket 370, drain port 324 of cap 312 and drain line 384. In this step, spent fluid from the patient's peritoneum is gravity fed through the patient's transfer set, patient line 382, patient port 360, drain port 324 and drain 384 to a drain bag connected to drain line 384.

In FIGS. 32B and 33B, a second step is a flush and a prime step. Here, patient line cap 350 is rotated ninety degrees relative to two line cap 312, such that patient line 382 and port 360 of cap 350 come into alignment with blind flange 376b of gasket 370. Blind flange 376b and wall 380 of gasket 370 close patient line 382. Seal 376b seals to underneath wall 362 of patient line cap 350. In this same step, slot 364 of cap 350 comes into fluid communication with drain nipple 374 and solution nipple 378 of gasket 370 to allow fresh solution from solution line 388 after frangible 386 is broken (FIGS. 33A to 33E do not show frangible 386 being broken for ease of illustration) to flow through solution port 328 of cap 312, solution nipple 378 of gasket 370, slotted pathway 364 of cap 350, drain nipple 374 of gasket 370, drain port 324 of cap 312 and drain line 384 to the drain bag. The drain line is then flushed and primed.

In FIGS. 32C and 33C, a third step is a filling step. Here, patient line cap 350 is rotated in the same direction ninety degrees, so that patient line 382 and port 360 of cap 350 are rotated into alignment with raised seal 376c and solution nipple 378 of gasket 370, solution port 328 of cap 312, and solution line 388. In this same step, cap 350 is rotated such that drain line 384 and corresponding gasket nipple 374 are sealed via raised seal 376a and wall 380 of gasket 370. Seal 376a seals to the inside or underneath wall 362 of patient line cap 350. In step three, fresh solution gravity flows through solution line 388, caps 312 and 350, through patient line 382, through the patient's transfer set and into the patient's peritoneum. The patient fill is performed until the patient's peritoneum is full and/or the solution bag is emptied.

As seen in FIGS. 32D and 33D, in a fourth step cap 350 is rotated forty-five degrees in the same direction from the position of step three to an all-lines-closed position. This position corresponds to the patient dwell, in which the fresh solution just introduced into the patient's peritoneum is allowed to reside in the peritoneum for a prescribed treatment time. Here, patient line 382, drain line 384 and solution line 388 are all blocked or closed. The area of surface 362 around patient port 360 of cap 350 comes into sealing engagement with blind or raised seal 376d. Raised seals 376a and 376c associated with drain line 384 and solution line 388, respectively, come into sealing engagement with surface 362 of cap 350 to block or close those lines.

In step five shown in FIGS. 32E and 33E, cap 350 is rotated again forty-five degrees to an all-lines-open state. Once the patient disconnects patient line 382 from the transfer set, patient line 382 becomes a final drain line and the all-lines-open state allows any remaining fluid in a solution bag to run through solution line 388, through device 310 and patient line 382 to a house drain. Also, the previous spent solution now residing in the drain bag can flow through drain line 384, device 310 and patient line 382 to the house drain. As seen best in FIG. 32E, patient port 360 and patient line 382 are rotated away from either blind seal 376b or 376d. Slot 364 in turn communicates with drain and solution ports 324 and 328 and corresponding lines 384 and 388. Fluid can flow to slot 364 under wall 362 from patient port 360 or from slot 364 under wall 362 to patient port 360. In this configuration, fluid can flow through patient, solution and drain lines simultaneously. As seen in FIG. 33E, blind pathway 364 of patient cap 350 allows fluid in the solution and drain bags to drain through the respective lines, through blind slot 364 of cap 350 to patient line 382 and to house drain or drain container.

Referring now to FIGS. 34A to 34F, in the U.S., the first step is a vent step, which allows the drain line and the solution to communicate during a steam sterilization cycle. As discussed below in connection with FIGS. 42 to 45, the U.S. or North America places the frangible seal at the end of the solution line at the solution bag. Two ends of any line cannot be closed during steam sterilization because if so the line will likely close or partially close during the sterilization process. Accordingly, the U.S. version of device 310 begins with an all lines open or vented state in which all lines communicate fluidly with a drain bag so that air can move from the drain bag to the lines during steam sterilization to prevent the lines from collapsing. Europe and other places on the other hand place the frangible seal at the Y-connection such that fluid from the supply container fills the solution line preventing the line from collapsing during sterilization. Accordingly, device 310 for Europe and other places can be set initially in a patient drain state with the solution line clamped.

Drain line 384 and solution line 388 are sealed via raised seals 376a and 376c, respectively, of gasket 370 to the underside surface 362 of cap 350. Patient line 382 and patient port 360 of cap 350 reside slightly above top surface 380 of gasket 370 to allow for raised seals that reduce friction versus a continuous seal between gasket 370 and cap 350. The raised or volcano seals collapse and any remaining gap between gasket and cap 350 is primed before use. The remaining gap also allows for all the lines to communicate if this is desired.

In step two (drain) of FIG. 34B, patient line cap 350 is rotated forty-five degrees, such that patient port 360 and patient line 382 come into fluid communication with drain port 324 of dual line cap 312. All of the solution lines, blind seals and openings of gasket 370 are in the same drain position of FIG. 34B as shown in the drain position of FIGS. 32A and 33A. As with the European steps, the patient thereafter rotates cap 350 ninety degrees such that the cap comes into the flush and prime configuration in FIG. 34C, which corresponds to the position of device 310 in the European therapy of FIGS. 32B and 33B. Next, the patient rotates cap 350 again ninety degrees, such that in the fill step of FIG. 34D, lines of device 310 correspond to the fill position of FIGS. 32C and 33C. The U.S. all-lines-closed position of FIG. 34E (after a forty-five degree rotation) corresponds to the European all-lines closed position of FIGS. 32D and 33D. The U.S. all-lines-open position of FIG. 34F (after a forty-five degree rotation) corresponds to the European all-lines-open position of FIGS. 32E and 33E.

### Stopcock Flow Control Devices

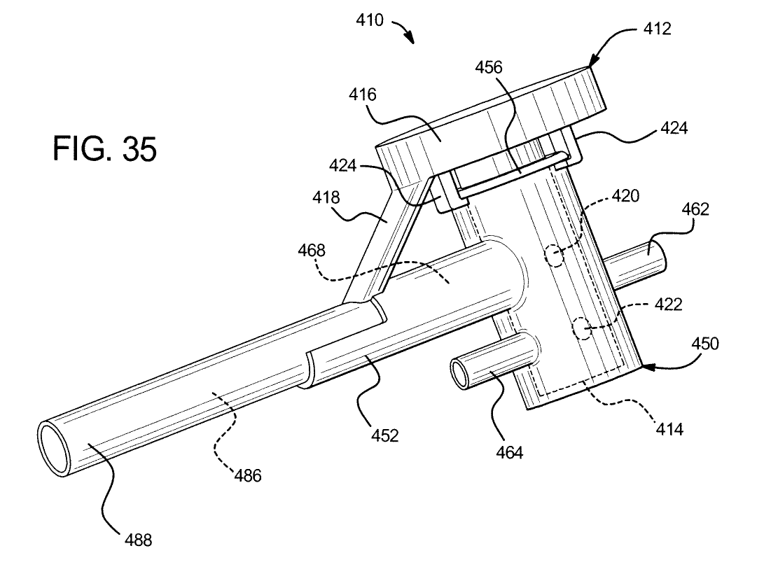

Referring now to FIGS. 35, 36, 37, 38A, 38B, 39, 40A to 40C and 41, a further alternative primary embodiment for a flow control device, which can be used for CAPD, for example, is illustrated via device 410. Device 410 includes a valve 412 and an outer housing 450. Valve 412 includes an inner cylindrical body 414 and a handle 416. A frangible tab 418 extends from handle 416 and locks valve 412 initially in place against a stem 452 of outer cylindrical housing 450. Stem 452 doubles to allow the patient to bend solution line 488 to break a frangible seal 486 within the solution line 488 to allow solution to flow through flow control device 410. Once frangible tab 418 is broken away from handle 416, the patient can twist handle 416 and corresponding valve body 414 into the next flow control position. Device 410 is made of any suitable material, e.g., any plastic discussed herein.

Besides stem 452, outer cylindrical housing defines a patient port 462 that is connected sealingly to a patient line (not illustrated) and a drain port 464 that is connected sealingly to a drain line (not illustrated). Inside stem 452, housing 450 includes or defines a solution port 468, which is connected sealingly to solution line 488 shown for reference in FIG. 35.

FIG. 35 illustrates one embodiment of fluid control device 410, in which valve body 414 defines lumens, such as lumens 420 and 422, that allow select ones of patient port 462, drain port 464 and solution port 468 to communicate with one another at different steps in the therapy. For example, in the European version, device 410 of FIG. 35 can be fixed initially in a drain position such that in the configuration of FIG. 35, a lumen (not illustrated) is defined by valve body 414, which allows patient port 462 to be in fluid communication with drain port 464. In the drain sequence, solution port 468 is sealed against body 414 of valve 412. For example, housing 450 can include volcano or raised ridges that project inwardly around ports 462, 464 and 468 to make positive contact with body 414 (alternatively, body 414 of valve 412 includes or defines outwardly extending volcano or raised ridges than encircle lumens 420 and 422). When the inwardly projecting volcano type apparatus moves over one of the apertures, such as aperture 420 or 422 in body 414, the lumen of the corresponding port comes into fluid communication with the corresponding lumen of valve 412.

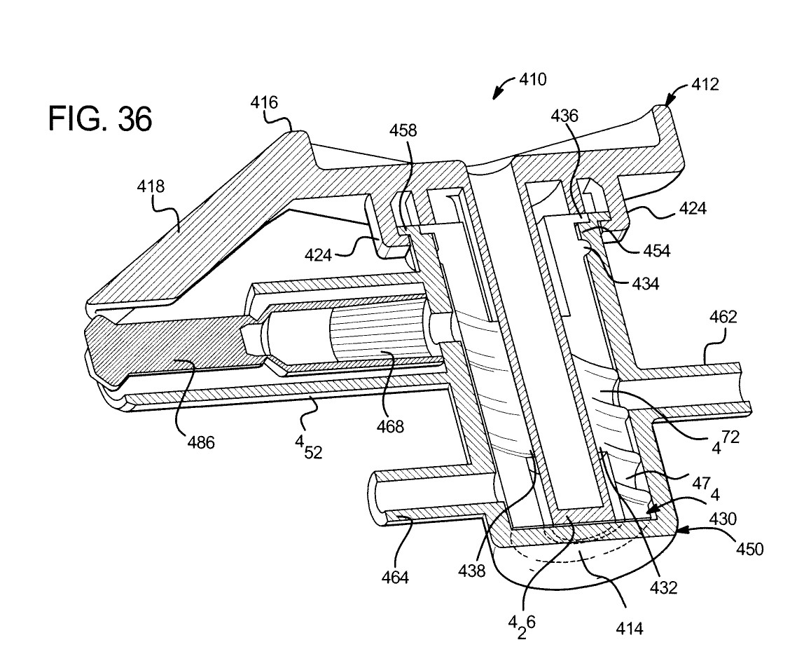

The lumens can be horizontal, diagonal, vertical, straight or curved. For example, two vertically disposed ports can be made to communicate via a curved “C” or “U” shaped lumen. The lumens can be defined by inner, thin-walled, e.g., molded, tubing sections or can be, e.g., molded or drilled, bores through a solid core body 414. FIGS. 36, 37, 38A, 38B, 39 and 40A to 40C discussed in detail below show an alternative embodiment in which raised seal flow paths are provided instead of lumen flow paths. The raised seals project outwardly from and along body 432 of a grommet 430 of valve 412 explained in more detail below.

In the example shown in FIG. 35, after breaking tab 418 and draining the patient via drain port 464 and patient port 462, the patient twists handle 416, e.g., counterclockwise, such that solution line port 468 comes into fluid communication with a valve body lumen beginning at opening 420, which extends in a semicircular manner to opening 422, which is in communication now with drain port 464 that has been rotated along with ports 462 and 468 of housing 450. The patient breaks frangible seal 486 and allows the solution to flow through solution line 488, solution port 468, opening 420, a lumen extending through to valve body 414 to opening 422, out drain port 464 and drain line (not illustrated) to flush and prime the drain line (frangible seal 486 provided at device 410 only in European version, North American version does not have frangible seal 486 provided at device 410 as discussed below).

After flush, the patient turns handle 416 in the same, e.g., counterclockwise direction to another set of openings (not illustrated), which allow solution port 468 and patient port 462 to communicate fluidly. Here, fresh solution flows via gravity through solution line 488, solution port 468, an internal lumen of housing 414 (not illustrated), out patient port 462 and a patient line (not illustrated) to the patient.

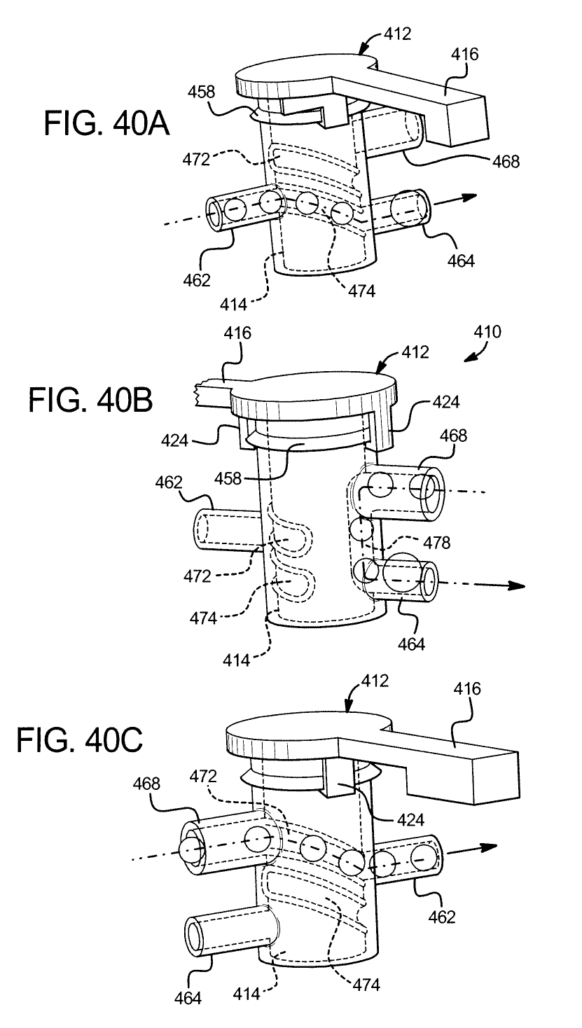

Each time handle 416 is turned, valve body snaps out of a held position and is turned to a new snap-fitted position. Flexible members 424 flex out of a holding position at an indent 456 and into a new holding position at a new indent 456 (see, e.g., FIG. 40 showing indents 456a to 456d). Tabs 424 and indents 456 operate to prevent overtravel and to hold valve 412 in a releasable position relative to housing 460, e.g., preventing unwanted reverse of valve 412 relative to housing 450.

After fill, handle 416 is turned again in the same direction. In the next position, each of the external projecting volcano type raised ridges surrounding ports 464, 468 and 462 come into sealing contact with body 414 of valve 412, to produce an all-valves-closed state during which the patient allows the newly injected dialysate to dwell within the patient's peritoneum. Alternatively, outwardly projecting ridges from body 414 around apertures 420 and 422 seal to an inner surface of housing 450.

Next, handle 416 is twisted in the same direction until flexible tabs 424 snap-fit into a next set of apertures 456, such that housing 450 and valve 412 are at an all-lines-open step, in which a lumen outputting to three openings, such as openings 420 and 422, enables fluid communication between each of patient port 462, drain port 464, and solution port 468. The all-lines-open position enables remaining fluid in the solution bag and the drain bag to drain out to a house drain via the patient line connected to port 462. As described above, this occurs after the patient removes the patient line from the patient's transfer set.

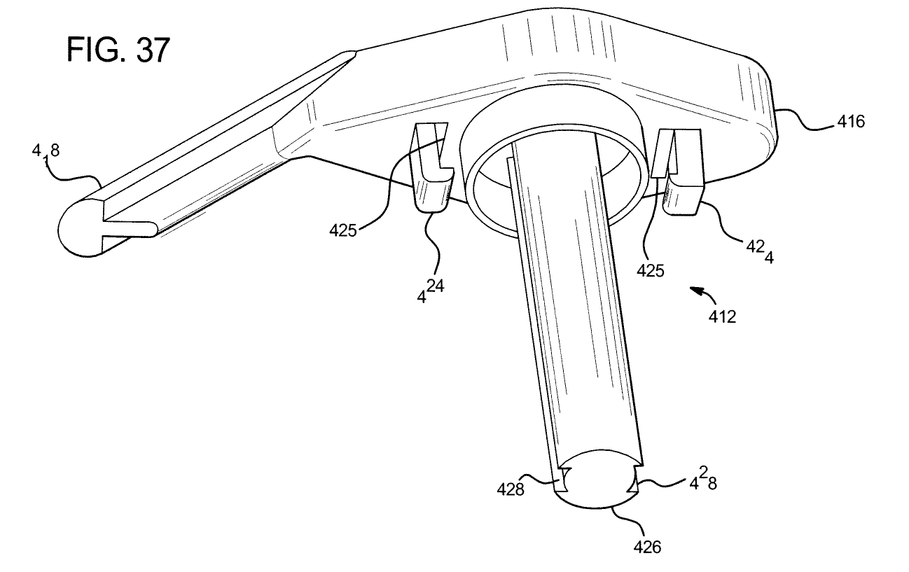

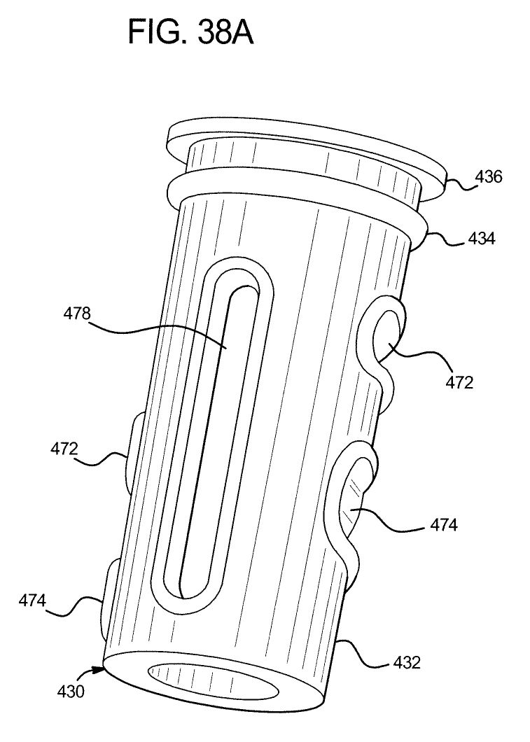

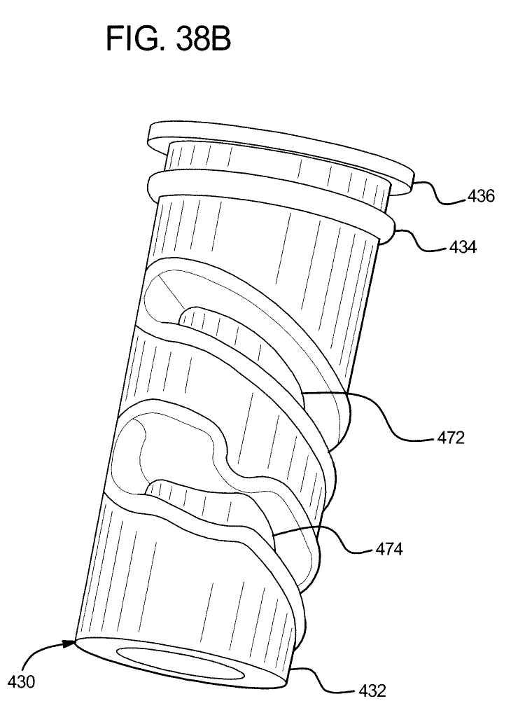

FIGS. 36, 37, 38A, 38B, 39 and 40A to 40C illustrate an alternative fluid control device 410. Here, valve 412 includes, e.g., connects to, a separate grommet 430 shown in detail in FIG. 36. Alternatively, valve 412 shown in detail in FIG. 37 is formed integrally or assembled with grommet 430. Grommet 430 shown in detail in FIGS. 38A and 38B defines or includes external flow paths, such as flow paths 472, 474 and 478, which replace (or are provided in addition to) the internal lumens shown in FIG. 35. Flow paths 472, 474 and 478 are formed via raised lips or ridges, which are formed with, adhered to, or are otherwise provided on the surface 432 of grommet 430.

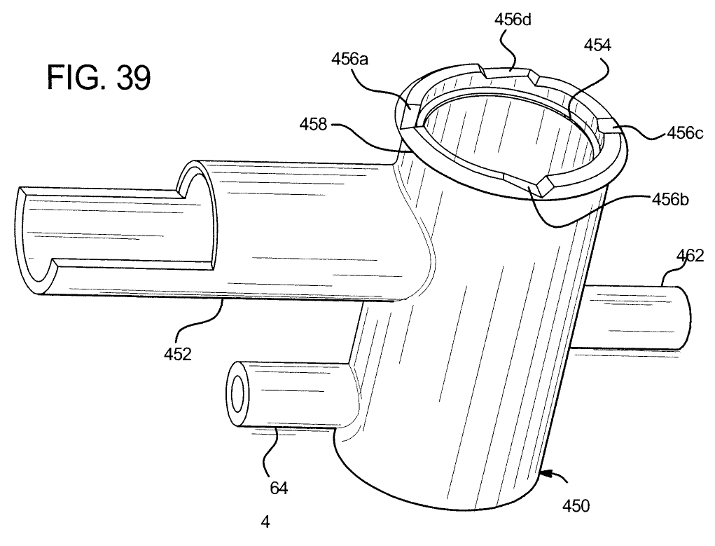

The raised lips of flow paths 472, 474 and 478 can be integral to and of the same material as grommet 430, or be made of a softer, more compliant material that is placed onto grommet body 432. The additional compliance of the material helps to form a rotating seal between the inside surface of housing 450 (shown in detail at FIG. 39) and the outside surface of grommet body 432. The paths, such as paths 472 and 474, spiral downwardly or upwardly, circumferentially around the outside of grommet body 432, so as to enable different ports of housing 450 to come into fluid communication with one another as valve handle 416 is turned within housing 450. The pathways can alternatively and/or additionally be horizontal or vertical, such as pathway 478.

FIGS. 36, 38A, 38B and 39 show that valve housing 450 defines an inwardly protecting sealing ring 454 that seals between outwardly projecting sealing ring 434 and upper rim 436 of grommet 430. The mating apparatuses connect grommet 430 to housing 450 and provide a seal between the two devices. Grommet 430 however can rotate with respect to housing 450.

FIGS. 36 and 37 illustrate that valve 412 includes a stem 426 that extends downwardly from handle 416 of valve 412. Stem 426 in turn defines grooves 428 that accept projections 438 (FIG. 36) extending inwardly from an inner surface of body 432 of grommet 430. In this manner, grommet 430 turns as valve 412 is turned.

As seen in FIGS. 36, 37 and 39, tabs 424 of valve 412 snap around an upper rim 458 of housing 450. Upper rim 458 of housing 450 in turn defines indents 456a to 456d. Indents 456a to 456d accept one or more snap-fitting apparatus 425 formed for example with one or more of tabs 424 to hold valve 412 and mated grommet 430 in a desired position with respect to housing 450. As seen, indents 456a to 456d include ramped indented surfaces for tactile feedback and a releasably locked connection with snap-fitting apparatus(es) 425 of valve 412.

Referring now to FIGS. 40A to 40C, drain, flush and fill sequences using device 410 are illustrated. It should be appreciated that a U.S. or North American version of device 410 would begin in an all-lines-open state because device 410 replaces Y-connection 190 shown FIGS. 15 to 19, for example. The U.S. version of device 410 is turned to the drain position of FIG. 40A. The European version of device 410 begins in the drain position of FIG. 40A.

In FIG. 40A, handle 416 of valve 412 is rotated such that the ends of diagonal pathway 472 come into fluid communication with drain port 464 and patient port 462. In the U.S. version, the patient turns handle 416 until snap-fitting apparatus(es) 425 move out of a home holding position and slide into snap-fitting contact with one or more of the apertures or indents 456a to 456d defined by or included with housing 450. This enables waste dialysate to gravity feed through patient port 462, down lower raised-lip, diagonal elliptical path 474, and out drain port 464.

When the patient has been drained completely, the patient in FIG. 40B and via handle 416 rotates valve 412 in the same direction, such that snap-fitting apparatus(es) 425 move out of the drain holding position and slide into snap-fitting contact with one or more of the apertures or indents 456a to 456d defined by or included with housing 450. Here, a vertical pathway 478 defined by a vertically disposed elliptical lip enables fresh solution to gravity flow through solution port 468, downwardly through vertical pathway 478 and out drain port 464, to flush the corresponding drain line.

Once flush is complete, the patient in FIG. 40C again turns handle 416 in the same direction until valve 412 snap-fits into the next one or more aperture 456a to 456d. This allows solution port 468 to communicate via upper raised-lip, diagonal elliptical path 472 with patient port 462. Here, fresh solution is gravity fed from the supply bag, through the solution line tube, through solution port 468, down path 472, out patient port 462, through the patient line, the patient's transfer set and to the patient.

In an embodiment, the raised ridge paths 472, 474 and 478 are combined with one or more internal pathways, such as the one described in connection with FIG. 35, to enable all three ports to communicate when valve 412 is rotated to an all-lines-open position. Here, grommet 430 defines the internal pathways in any manner described above in connection with valve body 414 of valve 412. It should be appreciated however that in any embodiment an all-valves-closed position can be provided in which all lines are closed. This is achieved via raised-ridge circular, blind seals (similar to blind seals 376 above) on the outer surface of grommet body 432, which seal around each of ports 462, 464 and 468 to an inner surface of housing 450. The raised seals seal the channels in the grommet and corresponding fluid paths.

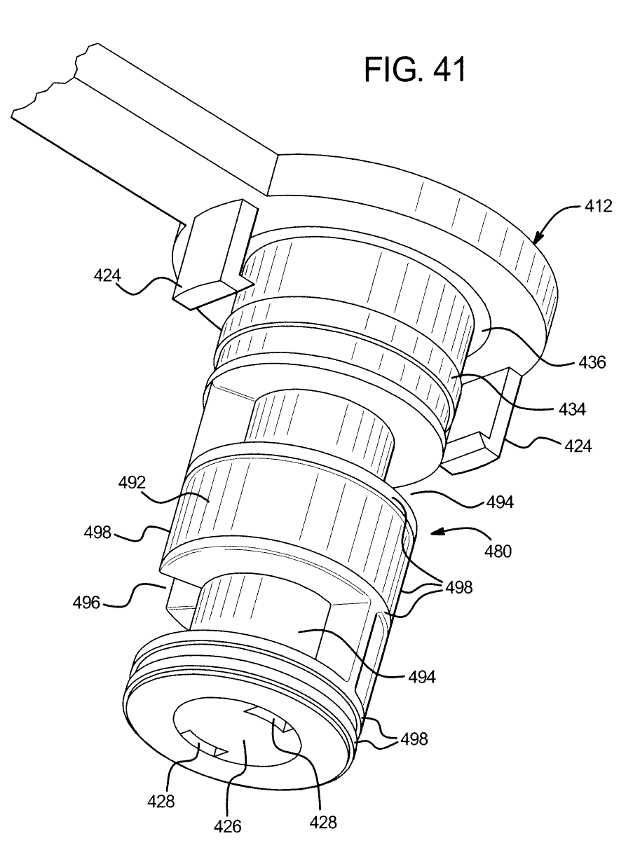

FIG. 41 illustrates an alternative grommet 480, shown for reference with valve 412. Valve 412 can include a stem 426 defining grooves 428 that accept projections 438 extending inwardly from an inner surface of body 492 of grommet 480 (as described above in connection with FIG. 36), such that grommet 480 rotates with valve 412. Grommet 480 includes generally horizontal grooves 494 and generally vertical grooves 496 connecting horizontal grooves 494. Grooves 494 and 496 can individually or in combination with each other extend from one port in mating housing 450 (not illustrated here) to another port of mating housing 450.

Body 482 defines or includes raised ridges 498 extending about vertical grooves 496, horizontal grooves 494, the bottom of grommet 480, or other place in which it is desired for grommet 480 to seal to an inner surface of housing 450. Raised ridges 498 can be integral with body 492 or be provided separately, e.g., as a softer or more compliant material. Grommet 480 also includes or defines seal ring 434 and upper rim 436 for connecting and sealing to housing 450 as described above in connection with FIG. 36.

Referring now to FIG. 42, placement and operation of the various CAPD flow control decreases described herein with a CAPD solution/tubing set 500 is illustrated. Set 500 is termed a European or “EU set” but may be used elsewhere besides the EU. Set 500 includes a supply bag 502, which is illustrated as a dual chamber supply bag, but is alternatively a single chamber supply bag or a supply bag having three or more chambers. Set 500 also includes a drain bag 504. Supply bag 502 is connected to a Y-connector 190 (shown above in FIGS. 15 and 16) via supply line 588 (representing any of the supply lines described herein, which each end with the number 88). Drain bag 504 is connected to Y-connector 190 via a drain line 584 (representing any of the drain lines described herein, which each end with the number 84). The patient line is connected to the patient and is not shown. The flow control devices described herein are placed at the section of set 500 corresponding to Y-connector 190.

Devices 10 (lever), 210 (twist activated) and 110 (dial activated) use a different Y-connector than Y-connector 190. FIG. 6 shows base 12 for device 10. The tubing set is not shown but it is apparent that the Y-connector for device 10 is configured such that the patient and drain lines split adjacently and in parallel (fitted into lumens 32 and 34) from the solution line (fitted into lumen 28). Devices 11 and 210 on the other hand configure the Y-connector such that the drain and solution lines split adjacently and in parallel from the patient line.

Devices 310 and 410 replace Y-connector 190 altogether. For example, FIG. 44 shows the replacement of Y-connector 190 with device 310. Y-connector 190 in FIG. 42 has a transfer set connector 194 and pull-cap 196 shown in more detail in FIG. 15. When devices 310 and 410 replace the Y-connector, transfer set connector 194 and pull-cap 196 are placed instead at the patient or distal end of a short patient line 382/482 extending from devices 310 and 410, respectively. FIG. 44 shows a short patient line 382 terminating with a transfer set connector 194 and pull-cap 196. The patient removes pull-cap 196 and connects transfer set connector 194 to the patient's transfer set, which in turn is connected to the patient.

In EU set 500, frangible seal 586 (representing all frangible seals discussed herein, each of which ends with the number 86) is placed at Y-connector 190, illustrated in FIGS. 15 and 16 for example. In those figures, however, tubing segment 192 is added so that frangible seal 186 is moved upstream towards bag 502 for operation with flow control device 110. Normally, as seen in FIG. 42, frangible seal 586 is placed directly adjacent to Y-connector 190. In situations in which devices 310 and 410 replace Y-connector 190, the frangible seal is placed (for EU operation) at the interface between the solution line and the flow control device as has been shown herein.

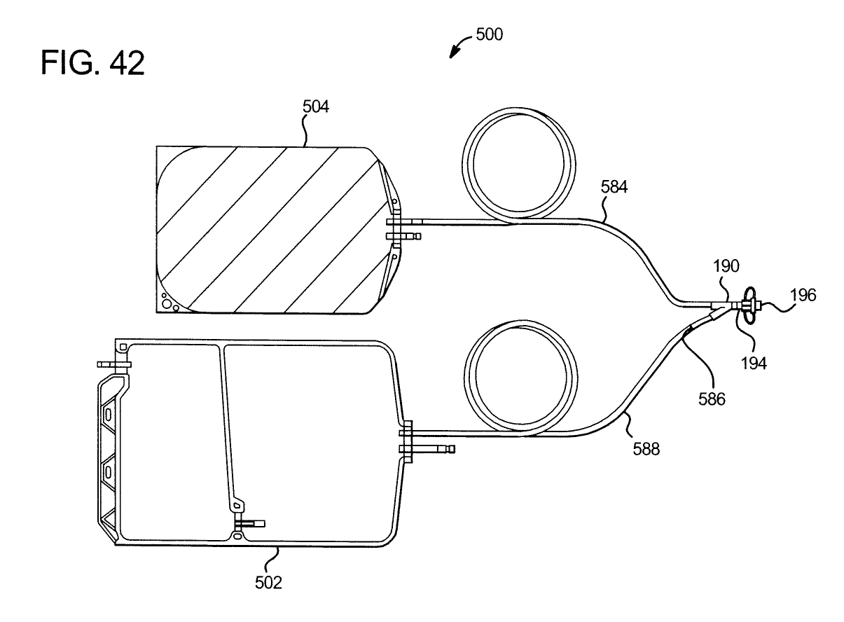

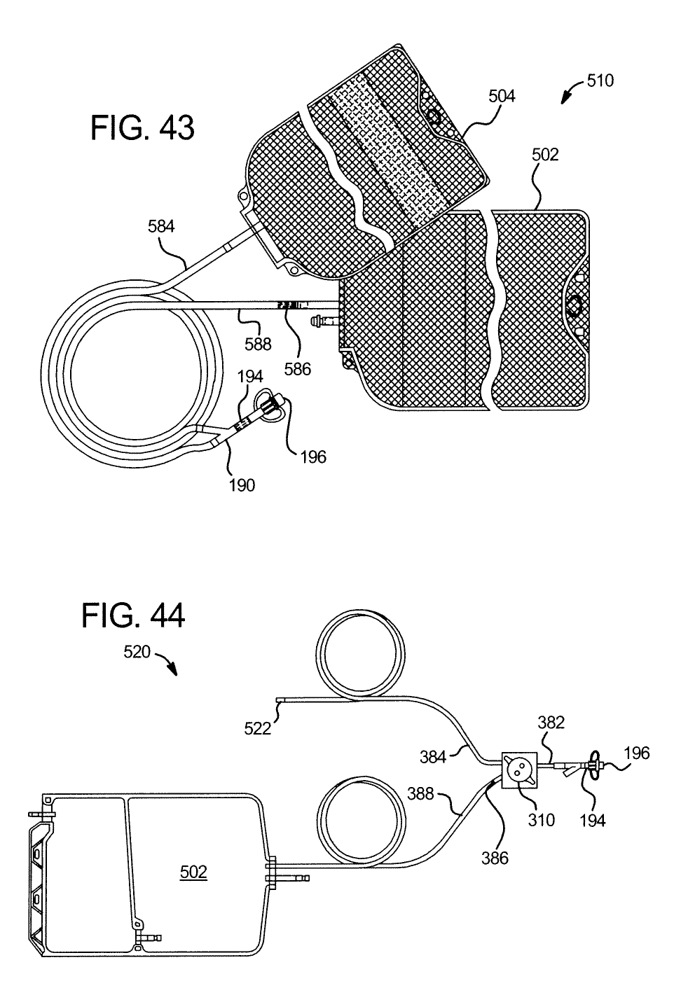

FIG. 43 shows a U.S. or North American set 510, which also can be used in different countries but for purposes of illustration is termed a U.S. set. U.S. set 510 includes supply bag 502, drain bag 504, supply line 588, drain line 584, Y-connector 190 and associated connector 194 and pull-cap 196. The main difference between EU set 500 and U.S. set 510 is the placement of frangible seal 586. U.S. set 510 places frangible seal 586 at supply container 502, preceding supply line 588. Accordingly, that end of supply line 588 is closed. If the flow control devices 310 and 410 are configured to initially occlude supply line 588 for a patient drain, both ends of supply line 588 would be occluded, which creates the collapsed tube potential during steam sterilization discussed above. Accordingly, the U.S. version of the flow control devices 310 and 410 are configured to initially not occlude either the supply or drain lines (or patient pigtail for devices 310 and 410), so that all lines can communicate with drain bag 504 during sterilization, pulling air from the drain bag if needed to prevent tubing collapse.

The EU set 500 does not experience such a problem because placing frangible seal 586 at the Y-connector 190 or device 310, 410 allows solution line 388, 488 or 588 to be filled with fluid at the time of sterilization, preventing collapse. Indeed, because devices 310 and 410 can be set initially in a drain mode, which occludes the solution line, it is contemplated to eliminate the frangible seal at the device. For example, devices 310, 410 can be provided with a frangible tab (e.g., tab 418 for device 410) that locks the flow control device into a solution line occluding position until the tab is broken. The locking device aids in the elimination of the frangible seal in the EU device versions.

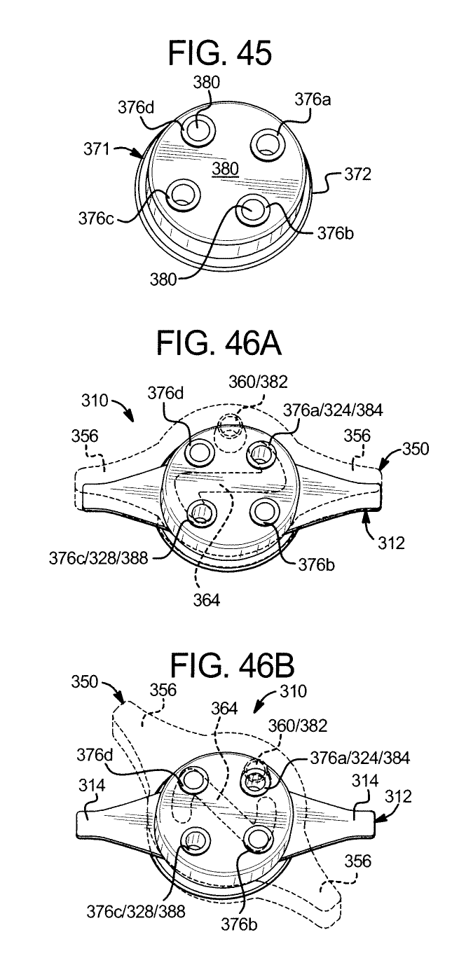

FIG. 44 illustrates an alternative set 520, in which drain bag 504 is eliminated. Removing drain bag from set 520 is advantageous for cost purposes. Set 520 is shown operating with device 310 and associated lines 382, 384, and 386 but can operate alternatively with any other device herein including device 410. FIG. 45 illustrates an alternative gasket 371 used for the eliminated drain bag flow control device. Comparing gasket 371 to gasket 370 of FIGS. 27A to 27C, it should be appreciated that raised or volcano seal 376d of gasket 371 is rotated away from seal 376c and towards seal 376a. Seal 376d of gasket 371 resides approximately one-hundred eighty degrees from seal 376b. Seal 376a for both gaskets 370 and 371 resides approximately one-hundred eighty degrees from seal 376c. The patient line cap of the eliminated drain bag flow control device can be the same as cap 350 of FIG. 31B.

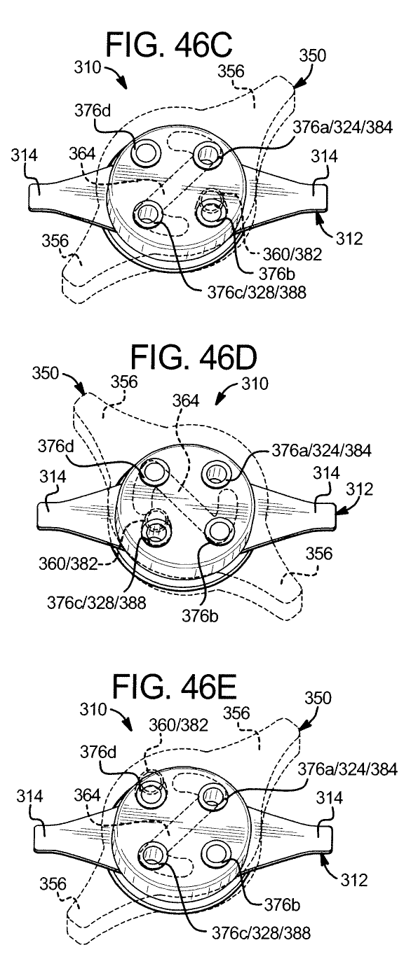

FIGS. 46A to 46E show one sequence of operation for a device 310 configured to eliminate the drain bag. In FIG. 46A, the sequence of device 310 is modified such that all lines 382, 384 and 388 are in fluid communication with each other during steam sterilization. Air is injected into supply bag 502 post filling to prevent tubing collapse and improve drainage. Solution line 388 is connected to the device. An, e.g., six inch patient tube 382 is then connected to device 310. Finally, drain line 384 is attached to the flow control device. Drain line 384 in one embodiment terminates with a non-vented tip protector 522.

During use, the patient:

1. rotates the flow control device 310 from the all lines open position of FIG. 46A to close the solution line 388 and open patient line 382 to drain line 384 as seen in FIG. 46B;

2. connects himself/herself to patient connector 194 at the end of line 382;

3. removes tip protector 522 from the free end of drain line 384 and places the drain line in a drain container or toilet;

4. opens the patient's twist clamp on transfer set line, allowing the patient to drain to the drain container or toilet;

5. rotates device 310 to allow the solution line 388 to open and flush and prime device 310 and purge any air from the system as seen in FIG. 46C;

6. rotates device 310 to occlude drain line 384 and infuse fresh solution to patient line 382 and the patient as seen in FIG. 46D; and

7. rotates device 310 to occlude patient line 382, preventing the solution line 388 and drain line 384 from draining during disconnect, as seen in FIG. 46E.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A peritoneal dialysis flow control device comprising:
a first cap including a first medical fluid line connection and a second medical fluid line connection;
a gasket mated with the first cap, the gasket defining a first aperture in fluid communication with the first medical fluid line connection and a second aperture in fluid communication with the second medical fluid line connection; and
a second cap including a third medical fluid line connection, the second cap sealed rotatably to the gasket mated to the first cap.

2. The peritoneal dialysis flow control device of claim 1, wherein at least one of the first, second and third medical fluid line connections includes a port configured to receive a medical fluid line.

3. The peritoneal dialysis flow control device of claim 1, wherein one of the gasket and the second cap includes a double-ribbed projection and the other of the gasket and the second cap includes single-ribbed projection, the single-ribbed projection fitting sealingly and rotatably between ribs of the double-ribbed projection.

4. The peritoneal dialysis flow control device of claim 1, wherein one of the first cap and the second cap includes a locking device and the other of the first cap and the second cap includes at least one locking feature, the locking feature mating with the locking device to releasably secure the second cap to the first cap at a desired relative position.

5. The peritoneal dialysis flow control device of claim 4, wherein the mating of the locking feature with the locking device is configured to provide at least one of: (i) tactile feedback; (ii) audible feedback; (iii) overtravel protection; and (iv) anti-reverse protection.

6. The peritoneal dialysis flow control device of claim 1, wherein the gasket defines at least one of: (i) at least one blind seal to seal the third medical fluid line connection when the third medical fluid line connection is rotated into alignment with the blind seal; and (ii) at least one blind passageway, a portion of the passageway communicating with the third medical fluid line connection when the third medical fluid line connection is rotated into alignment with the blind passageway portion.

7. The peritoneal dialysis flow control device of claim 1, wherein the second cap defines at least one blind passageway, the passageway communicating with at least one of the first and second medical fluid line connections when the at least one medical fluid line connection is rotated into alignment with the blind passageway.

8. The peritoneal dialysis flow control device of claim 1, wherein at least one of: (i) the first medical fluid line connection is a solution port; (ii) the second medical fluid line connection is a drain port; (iii) the third medical fluid line connection is a patient port; and (iv) one of the first and second medical fluid line connections cooperates with an apparatus positioned to aid a user to break a frangible seal in a line connected to the first or second medical fluid line connection.

9. The peritoneal dialysis flow control device of claim 1, wherein at least one of the first and second caps includes a grasping apparatus sized and shaped to enable a user to rotate one of the first and second caps relative to the other of the first and second caps.

10. A peritoneal dialysis flow control device comprising:
a first cap including a solution line connection and a drain line connection; and
a second cap including a patient line connection, the second cap sealed rotatably to the first cap so as to enable
a first relative position of the second cap to the first cap, in which the patient line connection is in fluid communication with the drain line connection,
a second relative position of the second cap to the first cap, in which the solution line connection is in fluid communication with the drain line connection, and
a third relative position of the second cap to the first cap, in which the solution line connection is in fluid communication with the patient line connection.

11. The peritoneal dialysis flow control device of claim 10, which includes at least one of: (i) an initial position in which gas can be vented from within the first and second caps when the first and second caps are assembled; (ii) an additional relative position, in which the none of the solution, drain and patient line connections is in fluid communication with each other; and (iii) an additional relative position, in which each of the solution, drain and patient line connections is in fluid communication with another of the solution, drain and patient line connections.

12. The peritoneal dialysis flow control device of claim 10, wherein at least one of: (i) the solution line connection is blocked in the first relative position of the second cap to the first cap; (ii) the patient line connection is blocked in the second relative position of the second cap to the first cap; (iii) the drain line connection is blocked in the third relative position of the second cap to the first cap; and (iv) the flow control device is configured to operate with a disposable set without a drain bag.

13. The peritoneal dialysis flow control device of claim 10, which includes at least one of: (i) a gasket, the gasket sealing the second cap rotatably to the first cap; and (ii) a configuration such that the second cap is rotated in a same direction between the first and second relative positions and second and third relative positions.

14. A peritoneal dialysis flow control device comprising:
a first cap;
a second cap sealed rotatably to the first cap;
a solution line connection, a drain line connection and a patient line connection provided with the first and second caps;
a first relative position of the second cap to the first cap, in which the patient line connection is in fluid communication with the drain line connection;
a second relative position of the second cap to the first cap, in which the solution line connection is in fluid communication with the drain line connection; and
a third relative position of the second cap to the first cap, in which the solution line connection is in fluid communication with the patient line connection.

15. The peritoneal dialysis flow control device of claim 14, wherein the first cap includes the solution line connection and the drain line connection, and the second cap includes the patient line connection.

16. The peritoneal dialysis flow control device of claim 14, which includes a forth relative position, in which none of the solution, drain and patient line connections is in fluid communication with another of the solution, drain and patient line connections, and a fifth relative position, in which each of the solution, drain and patient line connections is in fluid communication with another of the solution, drain and patient line connections.

17. A peritoneal dialysis flow control device comprising:
a housing having first, second and third medical fluid line connections; and
a valve fitted rotatably inside the housing, the valve including
a first flow path configured to communicate with the first and third line connections
a second flow path configured to communicate with the first and second line connections, and
a third flow path configured to communicate with the second and third line connections.

18. The peritoneal dialysis flow control device of claim 17, wherein at least one of: (i) at least one of the first, second and third medical fluid line connections includes a port configured to receive a medical fluid line; (ii) the first medical fluid line connection is a drain port; (iii) the second medical fluid line connection is a solution port; (iv) the third medical fluid line connection is a patient port; (v) one of the first and second medical fluid line connections cooperates with an apparatus positioned to aid a user to break a frangible seal in a line connected to the drain or solution port; and (vi) the valve includes a body, at least one of the first, second and third flow paths extending within the body; (vii) the housing includes an inwardly projecting seal around a mouth of at least one of the first, second and third medical fluid line connections, the seal configured to seal to the body about a mouth of the at least one flow path; (viii) the valve includes a body, at least one of the first, second and third flow paths extending along an external surface of the body; and (ix) a configuration such that the housing can be rotated with respect to the valve so that none of the first, second and third medical fluid line connections can communicate fluidly with any of the first, second and third flow paths

19. The peritoneal dialysis flow control device of claim 18, the body being one of: (i) at least substantially solid, the at least one flow path bored through the body; and (ii) at least substantially hollow, the at least one flow path formed as a tube extending through the body.

20. The peritoneal dialysis flow control device of claim 18, the at least one flow path including at least one of: (i) a continuous raised ridge forming a seal with an inner wall of the housing; and (ii) a configuration extending diagonally or vertically along the external surface of the body.

21. The peritoneal dialysis flow control device of claim 17, the valve configured such that the valve is rotated in a same direction (i) from a position in which the first flow path is in communication with the first and third line connections to a position in which the second flow path is in communication with the first and second line connections, and (ii) from the position in which the second flow path is in communication with the first and second line connections to a position in which the third flow path is in communication with the second and third line connections.

22. The peritoneal dialysis flow control device of claim 17, the valve configured such that the valve can be rotated in sequence (i) to a position in which the first flow path is in communication with the first and third line connections (ii) to a position in which the second flow path is in communication with the first and second line connections, and (iii) to a position in which the third flow path is in communication with the second and third line connections.

23. The peritoneal dialysis flow control device of claim 17, wherein the housing and valve include mating apparatuses that are configured to provide at least one of: (i) tactile feedback; (ii) audible feedback; (iii) overtravel protection; and (iv) anti-reverse protection.

24. The peritoneal dialysis flow control device of claim 17, wherein the valve includes at least one of: (i) a handle; and (ii) a grommet, the grommet forming the first, second and third flow paths.

25. A peritoneal dialysis flow control device comprising:
a housing having a plurality of medical fluid line connections; and
a valve fitted rotatably inside the housing, the valve including a body and a plurality of flow paths extending within the body, at least one of the flow paths bending 180 degrees to enable an inline pair of the medical fluid line connections existing on a same side of the housing to communicate fluidly.

26. The peritoneal dialysis flow control device of claim 25, the body being one of: (i) at least substantially solid, the flow paths bored through the body; and (ii) at least substantially hollow, the flow paths formed as tubes extending through the body.

27. The peritoneal dialysis flow control device of claim 25, the housing including an inwardly projecting seal around a mouth of each of the medical fluid line connections, the seals configured to seal to the body about a mouth of at least one of the flow paths.

28. A peritoneal dialysis flow control device comprising:
a housing having a plurality of medical fluid line connections; and
a valve fitted rotatably inside the housing, the valve including a body and a plurality of flow paths along an external surface of the body.

29. The peritoneal dialysis flow control device of claim 28, the flow paths each including a continuous raised ridge forming a seal with an inner wall of the housing.

30. The peritoneal dialysis flow control device of claim 28, wherein one of the flow paths extends diagonally and another vertically along the external surface of the body.

31. The peritoneal dialysis flow control device of claim 28, the valve including a grommet, the grommet forming the external surface of the body.
